(19) United States
(12) Patent Application Publication
(10) Pub. No.: US 2026/0305924 A1
(43) Pub. Date: Oct. 8, 2026

(54) LARGE BORE SUCTION OVERTUBE

(71) Applicant: Neptune Medical Inc., Burlingame, CA (US)

(72) Inventors: Jessica KOCH, San Francisco, CA (US); Wei Li FAN, San Francisco, CA (US); Kyle IMATANI, Mountain View, CA (US); Niraj A. SHAH, Palo Alto, CA (US); Elias M. ELEFTHERIADES, Redwood City, CA (US)

(21) Appl. No.: 19/481,880

(22) PCT Filed: May 6, 2024
(86) PCT No.: PCT/US2024/028047

§ 371 Date: Nov. 5, 2025

Related U.S. Application Data
(60) Provisional application No. 63/500,343, filed on May 5, 2023.

(51) Int. Cl. A61B 1/015; A61B 1/00; A61B 1/12; A61B 1/31; A61M 1/00; A61M 25/06

(52) U.S. Cl. CPC A61B 1/015; A61B 1/00078; A61B 1/00091; A61B 1/00154; A61B 1/126; A61M 1/77; A61M 1/87; A61B 1/31; A61M 2025/0681

(57) ABSTRACT

Large-bore suction overtube apparatuses including an inner lumen that is configured to receive an endoscope and to apply suction around the endoscope in order to remove material from within a body. In some cases the endoscope is removed, and in other cases removal of the endoscope is not required. Also described herein are methods.

### CLAIM OF PRIORITY

This patent application claims priority to U.S. Provisional patent application No. 63/500,343, titled “LARGE BORE SUCTION OVERTUBE,” and filed on May 5, 2023, herein incorporated by reference in its entirety.

### BACKGROUND

Devices that capture and retrieve thrombus and debris from the GI tract or other body regions may include aspiration catheters and may be used for a variety of procedures that may be performed with less invasive approaches to reach distant locations within a patient's body. These procedures may include entry into the patient's gastrointestinal system, vascular system, abdominal cavity, lungs, female genital tract, or into the urinary tract.

In some cases, it is beneficial to use a scope (generically referred to herein as an “endoscope”) to assist in removal of material, including blood clot material. Endoscopes may include one or more working channels and/or a suction channel and may be used to visualize clot material. In some cases, endoscopes may include mechanical means for assisting in the removal and/or disruption (e.g. macerating) clots. Furthermore, tools may be utilized through the working channel(s). However, the effectiveness of endoscopes may be limited, in part because of the dimensions of the suction channel and the size and organization of the material (i.e., clot may be too big and too firm relative to the working channel diameter). Other examples of problems with current technology include the time required or even the ability to access the occlusion site due to endoscope looping in the anatomy. There is a need for apparatuses and methods that allow for the rapid access and optimized aspiration of material (e.g., clot material, feces, food, etc.) from within the body. The apparatuses and methods described herein may fully address these issues.

### SUMMARY OF THE DISCLOSURE

Described herein are methods and apparatuses (e.g., devices, accessories, systems, etc., including suction overtubes) for removing material (e.g., stool, blood, blood clots, debris, wash fluid, etc.) from within a body. These removal apparatuses may include a suction overtube that is adapted for effective and intuitive use with one or more scopes (e.g., endoscopes). These methods and apparatuses may be referred to as suction overtubes, and may generally include an elongate body, which may be highly flexible but reinforced (to withstand a range of applied suction) and a proximal end that is adapted to receive an endoscope into the lumen of the overtube while allowing the endoscope to move longitudinally (proximally/distally) within lumen, while maintaining a sufficient seal so that suction can be applied at the proximal end to provide suction at the distal end of the overtube. The overtubes described herein may be configured to have the same stiffness or different relative stiffnesses. In some examples the apparatus may be configured to have a variety of different stiffnesses along the length of the overtube. For example the distal end regions may be progressively more flexible than the proximal end regions.

For example, a suction overtube apparatus may include: an elongate body having an inner lumen that extends from a distal end to a proximal end; a proximal end region including an endoscope receiving port configured to receive an endoscope therethrough into the inner lumen, wherein the endoscope receiving port is in line with the inner lumen of the elongate body; a vacuum port at the proximal end region in fluid communication with the inner lumen; and a control that controls flow into the vacuum port and configured to apply suction through the vacuum port when the control is held actuated by a user.

In some examples a suction overtube apparatus may include: an elongate body having an inner lumen that extends from a distal end to approximal end, the elongate body having a hoop strength sufficient to withstand negative pressure within the inner lumen (e.g., at least about 760 mmHg or pressure, at least about 700 mmHg, at least about 600 mmHg of pressure, at least about 500 mmHg, at least about 380 mmHg, at least about 300 mmHg, etc.); a proximal end region including an endoscope receiving port configured to receive an endoscope therethrough into the inner lumen and comprising one or more seals configured to seal around the endoscope, wherein the endoscope receiving port is in line with the inner lumen of the elongate body; a vacuum port at the proximal end region in fluid communication with the inner lumen; and a control coupled to the vacuum port and configured to apply suction through the vacuum port when the control is held actuated by a user. In general, the overtube may be configured to withstand various levels of vacuum, including varying levels of partial vacuum (e.g., one full atmosphere of 760 mmHg).

As mentioned, the overtube apparatus may generally include a seal that is configured to seal around the endoscope; this seal may be proximal to the vacuum port. For example, the proximal end region may include one or more sealing gaskets that are configured to seal around the endoscope. The seal may be configured to permit the endoscope to move (e.g., slide) proximally and distally relative to the lumen of the overtube apparatus. In some examples the seal may be adjustable so that it may be relaxed, e.g., to allow the scope to be more easily moved proximally and distally, and may be activated or tightened, e.g., prior to and/or during the application of suction through the lumen of the overtube. The seal may be an annular seal, such as (but not limited to) an O-ring or an elastomeric seal that is flat and thin. Any of these seals may be lubricated or coated for reduced sliding drag.

In any of these apparatuses, the endoscope receiving port and the vacuum port may be part of an adapter configured to couple to the proximal end of the elongate body. For example, an adapter may be configured (via a friction fit, threading, luer lock, etc.) to a proximal end of the overtube apparatus, and may include a port for applying suction through the overtube lumen (optionally, in some examples the port may be positioned on a side of the proximal end region of the overtube or adapter), and a proximal end port for receiving the endoscope. The control may be associated with the vacuum port, and may be part of the adapter. The adapter may be removably attached. The adapter may include the seal configured to seal around the endoscope.

Alternatively, the apparatus may not include a separably adapter, but may be integrated, so that the endoscope receiving port and/or vacuum port are integrated together with the elongate body of the overtube (e.g., at a proximal end of the overtube).

In general, these apparatuses may be configured to withstand (i.e., not leak or not have structural collapse) at a target range of negative pressures. For example, the apparatuses may be configured to withstand up to 760 mmHg of negative pressure (e.g., up to 700 mmHg of negative pressure, up to 600 mmHg of negative pressure, up to 500 mmHg of negative pressure, etc.). For example, the elongate body may have a hoop strength sufficient to withstand at least the target range of pressure (e.g., up to about 760 mmHg of negative pressure, 700 mmHg of negative pressure, 600 mmHg of negative pressure, 500 mmHg of negative pressure, 400 mmHg of negative pressure, etc., the device may be configured to withstand about 0.2 atm, 0.4 atm, 0.6 atm, 0.8 atm, or a full atm or more) within the inner lumen.

In any of these examples the elongate body may be reinforced. One or more layers of the elongate body forming the lumen may be a reinforced layer, including but not limited to a braided tube or coil-reinforced layer/tube. For example, the elongate flexible tube may comprise a coil-reinforced tube. The coil may be, e.g., a helical coli, formed of a material that exhibits a high compression and/or tensile strength. This could be a wire, a polymer, a composite fiber, a yarn made of a natural or man-made material (including reinforced with a stiff resin material, for example, an epoxy), a metal, a metal alloy, a composite material, a mineral, a polymeric material, a natural fiber, etc. In some cases it could be a fiber, including, for example, aramids (Kevlar, Twaron, Technora), Vectran, UHMWPE (Dyneema or Spectra), Zylon, nylon, polyester, or carbon fiber. In some cases it could be formed of a composite of multiple materials. In some instances it could be formed of a metal, including, for example nitinol, steel or plated steel, a stainless steel alloy, a magnesium alloy, tantalum, cobalt-chromium alloys, etc. The coil may be wound in a single direction or more than one direction (e.g., clockwise and/or counterclockwise). Thus, the elongate body may include an inner coil wound tube.

As mentioned, the apparatus may include one or more controls that may be coupled to the vacuum port to control the application of suction. For example, the control may be a button, slider, knob, valve, dial, lever arm, switch, trigger, etc. In some examples the control may be a biased valve. In general, the control may be biased to maintain the vacuum port in a closed position, so that no suction is applied unless the control is maintained in an activated state (e.g., maintaining force against the bias). In some cases the valve may be actuated directly, and in others it may be actuated electronically or remotely, and then actuated upon by an actuator (for example, a motor, rotary actuator, linear actuator, solenoid, etc.).

The control may be configured so that activation of the control seals (or increases the seal) around the endoscope. For example, the control may be coupled to the vacuum port and may include or incorporate a valve, such as but not limited to a trumpet valve. The control may be directly coupled to the vacuum port, or it may be coupled by a vacuum (e.g., suction) line coupled to the vacuum port. In some examples the control comprises a lever or petal (e.g., foot petal, hand lever, finger lever, etc.).

In general, the distal end of the overtube may be tapered. For example, the distal end of the overtube may have a diameter that is a fraction of the inner diameter of the more proximal region of the lumen of the overtube. For example, the diameter of the opening may be between about 97% and 50% of the inner diameter of the lumen of the overtube (e.g., less than 90%, less than 85%, less than 80%, less than 75%, less than 70%, less than 65%, less than 60%, etc.). Thus, the distal end opening of the overtube may be constricted.

The distal end opening of the overtube may have an inner diameter that is approximately the same as or larger than the outer diameter of the distal end of the endoscope. For example, the distal end opening of the overtube may have an open diameter that is approximately 5% larger than the outer diameter of the endoscope (e.g., 7.5%, 10%, 12.5%, 15%, 17.5%, 20%, 25%, 30%, 50%, 75%, 100%, 1.5×, 2×, 2.5×, 3×, 5×, etc. of the outer diameter of the endoscope). Any of these apparatuses may include (e.g., as part of a system) the endoscope.

In general, any of these apparatuses may have an inner lumen that has a cross-sectional area of greater than about 10 mm² (e.g., greater than about 12 mm², 15 mm², 20 mm², 25 mm², 30 mm², 50 mm², 70 mm², etc.).

The distal end of the overtube (e.g., the tip), including the distal tapered region surrounding the distal opening into the lumen, may be transparent and/or translucent. For example, the distal end of the overtube may be configured to allow visualization of the region of the body outside of distal end region (in some cases by the camera of the endoscope, when the endoscope is withdrawn into the lumen). In any of these apparatuses, the distal end region (including or proximal to the tip) of the overtube may include one or more release holes to prevent full suction, which may be helpful to prevent suctioning to the wall of the body, for example. The apparatus may include one or more (e.g., 2, 3, 4, 5, 6, 7, 8, 9, 10, etc.) holes or openings into the internal lumen through the apparatus. The suction relief holes may be smaller (individually and/or in aggregate) than the distal end opening of the apparatus. The suction relief holes may be positioned proximally of the distal end opening.

In any of the apparatuses described herein the overtube and/or the endoscope may be configured to rigidize. For example, the elongated body of the overtube may be configured to rigidize. The overtube may be configured to partially or completely rigidize; in some examples the overtube is configured so that just a region (e.g., the proximal region) may rigidize. In some examples the distal end portion (e.g., the distal-most ~4.5 inches, 5 inches, 6 inches, 7 inches, etc.) may be configured to not rigidize, but may remain flexible. This may allow the apparatus to be steered more easily over the steerable endoscope. In some examples the elongated body comprises a rigidizing layer comprising multiple strand lengths that cross over each other, and a compression layer that is configured to be actuated to apply force to the rigidizing layer to rigidize the rigidizing outer sheath from a flexible configuration to a rigid configuration. Examples of rigidizing apparatuses, the features of any of which may be included as part of the overtubes and/or endoscopes described herein may be found in U.S. Pat. No. 11,135,398 (titled “DYNAMICALLY RIGIDIZING COMPOSITE MEDICAL STRUCTURES”), U.S. patent application Ser. No. 17/604,203 (also titled “DYNAMICALLY RIGIDIZING COMPOSITE MEDICAL STRUCTURES”), PCT/US2021/024582 (titled “LAYERED WALLS FOR RIGIDIZING DEVICES”), PCT/US2021/034292 (titled “RIGIDIZING DEVICES”), PCT/US2022/014497, (titled “DEVICES AND METHODS TO PREVENT INADVERTENT MOTION OF DYNAMICALLY RIGIDIZING DEVICES”), PCT/US2022/019711, (titled “CONTROL OF ROBOTIC DYNAMICALLY RIGIDIZING COMPOSITE MEDICAL STRUCTURES”), U.S. Provisional Patent Application No. 63/265,934, (titled “METHODS AND APPARATUSES FOR REDUCING CURVATURE OF A COLON”), U.S. Provisional Patent Application No. 63/296,478, (titled “RECONFIGURABLE STRUCTURES”), U.S. Provisional Patent Application No. 63/308,044, (titled “DYNAMICALLY RIGIDIZING COMPOSITE MEDICAL STRUCTURES”), U.S. Provisional Patent Application No. 63/324,011, (titled “METHODS AND APPARATUSES FOR NAVIGATING USING A PAIR OF RIGIDIZING DEVICES”), U.S. Provisional Patent Application No. 63/342,618, (titled “EXTERNAL WORKING CHANNELS FOR ENDOSCOPIC DEVICES”), U.S. Provisional Patent Application No. 63/335,720, (“HYGIENIC DRAPING FOR ROBOTIC ENDOSCOPY”), and U.S. Provisional Patent Application No. 63/332,686, (titled “MANAGING AND MANIPULATING A LONG LENGTH ROBOTIC ENDOSCOPE”), each of which is herein incorporated by reference in its entirety.

Any of these apparatuses may include a source of negative pressure (e.g., vacuum), such a pump or the like. The apparatus may include control circuitry (e.g., a controller, including hardware, software and/or firmware for controlling suction, such as a suction manifold with one or more valves). Alternatively or additionally, the apparatus may be configured to operate using wall suction (e.g., centrally applied suction). The apparatuses described herein may include a suction trap (e.g., to capture liquid and/or solids, including one or more filters to filter and remove solids from the liquids that are aspirated by the overtube. The apparatus may include tubing.

Any of these apparatuses may be configured to apply positive pressure. For example, any of these apparatuses may be configured to apply positive pressure to unclog the lumen of the overtube in some examples. The control circuitry may be configured to control the application of positive pressure (e.g., for unclogging) in addition to negative pressure.

Any of these apparatuses may be configured or adapted to couple to apply suction through one or both of the lumen of the overtube and/or a suction lumen of the endoscope.

For example, the apparatus may include connection and controls to couple to the vacuum port of the overtube and an endoscope suction tube.

Also described herein are methods of removing material from within a body using any of the apparatuses described herein. For example, a method of removing a material from within a body may include: advancing an overtube and an endoscope distally within the body, wherein the endoscope is inserted through a proximal end port of the overtube into a lumen of the overtube so that a proximal end of the overtube is movably sealed around the endoscope; positioning a distal end of the overtube adjacent to a material to be removed; and applying suction through the lumen of the overtube from a port at a proximal end of the overtube (e.g., optionally on a side of the distal region of the overtube or an adapter on the overtube) while a user activates a control to draw the material around into the overtube and around the endoscope.

For example, a method of removing a material from within a body may include: inserting an endoscope through a proximal end port of the overtube into a lumen of the overtube so that a proximal end of the overtube is movably sealed around the endoscope; positioning a distal end of the endoscope adjacent to a material to be removed; positioning a distal end of the overtube adjacent to the distal end of the distal end of endoscope; withdrawing the endoscope proximally into the overtube; and applying suction through the lumen of the overtube from a port at a proximal end of the overtube while a user activates a control to draw the material around into the overtube and around the endoscope. Suction could be applied within the overtube, within the working channel, separately, or both.

Any of these methods may include coupling a suction source to the port at the proximal end region of the overtube.

In some examples the methods described herein may use the endoscope at an initial position near (e.g., adjacent to) the material to be removed. The endoscope may use imaging integrated or associated with the endoscope to identify the material to be removed within the body; in some examples the endoscope may be extended distally from out of the distal end opening of the overtube. The apparatus may be advanced with the endoscope extending distally out of the overtube, or the endoscope may be separately advanced distally and the overtube may be separately advanced once the endoscope is positioned. Any combination of these movements may be performed: advancing the endoscope distally while the overtube remains more proximal; advancing both the endoscope and overtube together distally (or withdrawing proximally together), holding the endoscope relatively fixed (relative to the patient's body) and advancing (or withdrawing) the overtube relative to the endoscope.

In general, positioning the distal end of the overtube may comprise positioning the distal end of the endoscope adjacent to the material to be removed. Positioning the endoscope adjacent may mean placing the endoscope sufficiently near the material to be removed so that it may be aspirated into the overtube when suction is applied (e.g. within about 5 cm, about 4 cm, about 3 cm, about 2 cm, about 1 cm, etc.).

Any of these methods may include withdrawing the endoscope proximally into the overtube before applying suction through the port. For example, the endoscope may be withdrawn proximally so that it does not block the opening into the overtube lumen; in addition, this may advantageously allow visualization of the material as it is withdrawn into the lumen of the overtube using the camera of the endoscope. For example, the endoscope may be withdrawn into the lumen of the overtube so that the tip of the endoscope is withdrawn at least 1 mm or more (e.g., 2 mm or more, 3 mm or more, 4 mm or more, 5 mm or more, 6 mm or more, 7 mm or more, 8 mm or more, 9 mm or more, 10 mm or more, 15 mm or more, etc.). In any of these methods, the material may comprise a clot material. Other materials may also be removed (e.g. debris, etc.).

Thus, in any of these examples the material (e.g., clot material or other material) may be drawn into the lumen of the overtube by suction and may pass around the endoscope, which remains within the lumen of the overtube.

In addition, any of these apparatuses and methods of using them may be configured to apply suction on endoscope to pull material (e.g., clot) into the overtube, after which the user may release suction on the endoscope (either before or after beginning suction through the inner lumen of the overtube) to allow clot to be sucked up by pathfinder.

In some examples the apparatus may be configured to maintain the seal around the endoscope as the endoscope is moved proximally or distally within the overtube. For example, the method may include moving the endoscope axially within the lumen of the overtube without breaking the seal while positioning the overtube.

As mentioned, the methods described herein may include applying suction through the lumen of the overtube from the port at the proximal end region of the overtube. In general, the suction may be applied through the overtube (e.g., maintaining suction) while the user operates the control, e.g., while the user is continuously pressing or actuating the control, and stopping the application of suction when the user stops operating the control. In some examples the control may include a trumpet valve, thus, applying suction through the lumen of the overtube from the port at the proximal end region of the overtube while a user activates the control may comprise applying suction while the user activates a trumpet valve.

The overtube and/or the endoscope may be steerable, for example by mechanical steering (e.g., one or more pullwires, tendons, etc.), pneumatic steering, electrical steering, etc. In general, any of these methods may include steering the distal end region of the endoscope independently of the overtube.

Any of these methods may include rigidizing either or both the overtube and the endoscope. For example, any of these methods may include rigidizing the overtube. In some examples, advancing the overtube and the endoscope may comprise rigidizing the overtube while advancing and steering the endoscope distally from the overtube, de-rigidizing the overtube, and advancing the overtube distally over the endoscope.

Any of the apparatuses described herein may include or be part of a robotic system. For example, the overtubes and/or endoscopes (including steerable, and/or rigidizing, etc. overtubes and/or endoscopes inserted or insertable through the overtubes) may be controlled by a robotic system.

For example, described herein are methods of removing material from the body of a patient, the method comprising: positioning a distal end of a rigidizing overtube adjacent to the material with an endoscope extending distally from distal end, wherein the rigidizing overtube concentrically surrounds a length of the endoscope so that the overtube and endoscope form an annular lumen; retracting a distal end of the endoscope proximally into a distal end region of the rigidizing overtube; and applying suction through the annular lumen to aspirate the material into the annular lumen and around the endoscope.

For example, described herein are methods of removing a material from the body of a patient, the method comprising: positioning a distal end of an endoscope adjacent to the material; positioning a distal end of an overtube extending over the endoscope adjacent to the distal end of the distal end of endoscope with the overtube in a flexible configuration so that the endoscope transitions from an extended configuration extending distal to the overtube to a retracted configuration, wherein the endoscope distal end is recessed within the distal end region of the overtube, so that the overtube concentrically surrounds the endoscope forming an annular lumen; converting the overtube from the flexible configuration to a more rigid configuration; and applying suction through the annular lumen to aspirate the material into the annular lumen and around the endoscope.

A method of removing a material from the body of a patient may include: positioning a distal end of an endoscope adjacent to the material; advancing a distal end of an overtube distally over the endoscope so that the distal end of the overtube is adjacent to the material while the overtube is in a flexible configuration, and positioning the distal end of the endoscope within a distal end region of the overtube to allow visualization out of the distal end of the overtube by the endoscope, so that the overtube concentrically surrounds the endoscope forming an annular lumen; converting the overtube from the flexible configuration to a more rigid configuration; and applying suction through the annular lumen to aspirate the material into the annular lumen and around the endoscope.

Any of these methods may include converting the rigidizing endoscope from a more flexible configuration when positioning the distal end adjacent to the material, to a more rigid configuration before retracting the endoscope proximally into the distal end region of the rigidizing overtube. The use of rigidizing overtubes may provide numerous benefits, including increasing the stability for the overtube which may enhance both visualization and the efficiency of the removal of material. For example, material may be removed by applying fluid through the endoscope for both general and focal removal of material. In a more rigid/less flexible configuration (referred to generally herein as a rigid configuration), the applied spray may be held steady, permitting focal application of wash fluid, even at lower fluid pressures, which may more effectively remove/wash the material.

In general, these methods may allow visualizing from the endoscope while applying suction to remove material. Visualization may be performed by one or more optics sub-systems/systems in the endoscope and/or the overtube. In some examples the endoscope may be used to visualized the distal end region of the overtube and into the body lumen (looking through the overtube) as suction is applied and/or as fluid (e.g., wash fluid) is applied.

The methods and apparatuses described herein may apply suction through the annular lumen formed between the overtube and the inserted endoscope. When applying suction, in some cases the endoscope may be retracted proximally so that the distal end of the endoscope (and thus the camera, working channel, etc.) is held recessed within the distal end region of the overtube (e.g., within the distal 10 cm, 9 cm, 8 cm, 7 cm, 6 cm, 5 cm, 4 cm, 3 cm, 2 cm, 1 cm, 5 mm, etc. and more preferably the distal 3 mm to 4 cm) of the overtube suction lumen. This position may be manually set (e.g., by the user moving the overtube relative to the endoscope and/or the endoscope relative to the overtube), or may be automatically set, e.g., by including one more engagers (e.g., detents, locks, etc.) between the overtube and the endoscope. The proximal seal between the endoscope and the overtube, e.g., at the proximal end region, may maintain the spacing, as the seal may provide some resistance between the endoscope and the overtube.

In general, the annular lumen around the endoscope and within the overtube is particularly and surprisingly more effective at removing material, including fecal matter, clot material, etc., which may otherwise clog more traditional aspiration lumen, including the suction lumen of the endoscope, as it may help shear and break up material. Further, the recessed end of the endoscope within the suction lumen (particularly where the distal end opening of the overtube is tapered inward) may further assist in breaking removing material and preventing clogging. The recessed position of the distal end of the endoscope both allows visualization (e.g., continuous visualization) and helps form the suction flow, modify the suction flow rate, and macerate material drawn into the overtube. In some cases the recessed position of the endoscope (e.g., between 3 mm and 4 cm) may generate turbulent flow within the suction opening into the overtube that may further assist in removal of material and preventing clogging. For example, any of these methods may be configured to include unclogging the annular lumen by repeatedly moving the endoscope distally and proximally within the overtube while apply suction through the annular lumen.

Irrigation may be applied from one or both of the overtube, the endoscope, and/or an irrigator extending through the endoscope and/or overtube. For example, irrigation fluid may be applied. Irrigation may be applied either before or after applying suction, e.g., through the annular lumen. Irrigation may be applied distal to the distal end of the overtube. For example, any of these methods may include applying irrigation by applying a spray of fluid from a fluid line of the overtube. In some cases, applying irrigation comprises extending an irrigator tube distally out of the distal end of the endoscope and applying irrigation into the body from the irrigator tube. The irrigator may comprise a radial irrigator. In some cases the irrigator may comprise a distal irrigator. Alternatively, in some cases, applying irrigation comprises switching between applying radial irrigation and distal irrigation by extending or retracting a radial irrigation tube relative to the distal end of the endoscope. Alternatively or additionally, in some cases, applying irrigation comprises applying irrigation through a fluid line integrated into the endoscope.

Any of these methods and apparatuses may include moving the endoscope axially within the overtube while applying suction. Moving the endoscope axially (e.g., distally and proximally) while applying suction may include moving the endoscope axially +/−between about 1 mm and 3 cm or more, e.g., between about 1 mm and about 3 cm, between about 1 mm and 2.5 cm, between about 1 mm and 2 cm, between about 1 mm and 1.5 cm, between about 1 mm and 1 cm, between about 1 mm and 7 mm, between about 1 mm and 5 mm, etc.). The endoscope may be moved back and forth in a linear oscillation, e.g., at between about 1 Hz and 50 Hz (e.g., between about 1 Hz and 40 Hz, between about 1 Hz and 30 Hz, between about 1 Hz and 20 Hz, between about 1 Hz and 10 Hz, etc.). Moving the endoscope within the lumen of the overtube while applying suction may help prevent or remove a clog. This linear movement of the endoscope relative to the overtube may be particularly effective and safe while the overtube is in a more rigid configuration (e.g., rigidized). This may provide stability allowing more precise movements, while protecting the body from harm.

In general, the methods and apparatuses described herein permit rotational movement of the endoscope within the lumen of the overtube. In some cases rotation of the endoscope within the overtube (particularly with the overtube in the rigid configuration) may enhance maneuverability of the endoscope while minimizing harm to the patient. In general, these methods may include preventing or removing clotting by rotating the endoscope within the lumen of the overtube (including rotational oscillation of the endoscope within the overtube. Linear and/or rotational movement of the endoscope relative to the overtube may be performed manually, semi-automatically and/or automatically, e.g., using mechanical assist.

Any of these methods may include moving the endoscope axially (e.g., distally/proximally) within the overtube while positioning the overtube. For example, when positioning the distal end of the overtube and/or the endoscope, either the endoscope or the overtube or both may be moved relative to each other. The endoscope may be steered independently of the overtube. For example, the method may include steering the distal end region of the endoscope independently of the overtube.

In some cases, positioning the overtube may include rigidizing the overtube while advancing and steering the endoscope distally from the overtube, de-rigidizing the overtube, and advancing the overtube distally over the endoscope.

Any of these methods may include sealing the proximal end of the overtube around the endoscope using a seal that allows the endoscope to advance/retract distally through the overtube lumen (e.g., movably sealed) while maintaining the suction/vacuum within the lumen of the overtube.

The methods and apparatuses described herein may be configured so that the cross-sectional area of the annular lumen (the suction annulus) may be between about 40 mm² and 400 mm² (e.g., between about 40 mm² and 300 mm², between about 40 mm² and 200 mm² , between about 40 mm² and 150 mm² , etc., between about 40 mm² and 100 mm², etc.).

Any of these methods may include positioning the overtube within the patient's gastrointestinal tract, including upper GI tract (mouth esophagus, stomach, pylorus, bile duct and pancreatic duct, etc.), the small bowel (e.g., small intestine, duodenum, jejunum, ilium, etc.), and/or the lower GI tract (rectum, regions of colon, e.g., sigmoid, descending, transverse, ascending, cecum, ileocecal valve, etc.). These methods and apparatuses may be configured to remove material such as but not limited to: fecal matter, blood, wash fluid, food debris, etc.

The methods and apparatuses described herein may also be configured to aid in uncoupling the apparatus (e.g., the overtube) from a wall of the body to which it may become coupled when operating the device. For example, when applying suction through the annular lumen the overtube may suck onto the wall of the body lumen. It may be particularly advantageous to quickly reduce or eliminate suction through by releasing the negative pressure (suction) within the annular lumen. For example, any of these methods may include releasing the overtube from a wall of the body by opening an air relief valve in fluid communication with the annual lumen while applying suction through the annular lumen. An air relief valve may a coupled to the inner lumen of the overtube distal to the suction port, and may be operated in conjunction with the application of suction, to titrate or release suction.

Also described herein are apparatuses (e.g., systems, devices, etc.) for performing any of these methods. For example, described herein are suction overtube apparatuses that may include: an elongate body having an inner lumen that extends from a distal end to a proximal end, wherein the elongate body is configured to convert between a flexible configuration to a more rigid configuration by the application or release of pressure within a wall of the elongate body; a rigidizer port (e.g., a pressure port) configured to receive positive and/or negative pressure to convert the elongate body from a flexible to a rigid configuration; a proximal end region including an endoscope receiving port configured to receive an endoscope therethrough into the inner lumen, wherein the endoscope receiving port is in line with the inner lumen of the elongate body; and a vacuum port at the proximal end region that is in fluid communication with the inner lumen.

For example, described herein are suction overtube apparatuses that include: an elongate body having an inner lumen that extends from a distal end to a proximal end, wherein the elongate body comprises a plurality of layers and is configured to transition from a flexible configuration to a more rigid configuration by the application of pressure to the plurality of layers; a proximal end region including an endoscope receiving port configured to receive an endoscope therethrough into the inner lumen, wherein the endoscope receiving port is in-line with the inner lumen of the elongate body; a rigidizer port (e.g., pressure port)configured to receive positive and/or negative pressure to rigidize the elongate body; a vacuum port at the proximal end region is in fluid communication with the inner lumen; and a control coupled to the vacuum port that is configured to apply suction through the inner lumen from the vacuum port when the control is actuated by a user.

A suction overtube apparatus may include: an elongate body having an inner lumen that extends from a distal end to a proximal end, the elongate body comprising a plurality of layers, wherein the elongate body has a hoop strength sufficient to withstand a vacuum of 760 mm Hg or greater, further wherein the elongate body is configured to transition from a flexible configuration to a more rigid configuration by the application of positive and/or negative pressure to the plurality of layers; a proximal end region including an endoscope receiving port configured to receive an endoscope therethrough into the inner lumen and comprising one or more seals configured to seal around the endoscope, wherein the endoscope receiving port is in line with the inner lumen of the elongate body; a rigidizer port (e.g., pressure port) configured to receive positive and/or negative pressure to rigidize the elongate body; and a vacuum port at the proximal end region in fluid communication with the inner lumen.

The distal end opening of the suction overtube may be tapered.

In general, the overtube may be rigidizing. For example, the elongated body of the overtube may include a rigidizing layer comprising multiple strand lengths that cross over each other, and a compression layer that is configured to compress the rigidizing layer in the more rigid configuration. Other structures for rigidizing may be used.

In general, these apparatuses may include a seal configured to seal around the endoscope proximal to the vacuum port. For example, the proximal end region of the overtube (or an adapter for an overtube) may include one or more sealing gaskets configured to seal around the endoscope.

Any appropriately sized overtube may be used. For example, the inner lumen of the overtube may have a cross-sectional area of greater than 10 mm² . In some case the overtube may be between 8 French and 32 French (or larger) (e.g., between 10 French and 24 French, etc.).

The apparatuses described herein, including the overtube, may be integrated or modular. For example, in some cases the endoscope receiving port and the vacuum port may be part of an adapter configured to couple to the proximal end of the elongate body of the overtube. Alternatively, the endoscope receiving port and the vacuum port (including the seal(s)) may be integrated into the overtube.

In general, these apparatuses may be configured and so that the overtube can withstand the inward (negative) pressure within the lumen of the overtube without collapsing or otherwise disrupting the function of the overtube (including in some examples, rigidizing). The elongate body may have a hoop strength sufficient to withstand at least 700 mm Hg (e.g., 750 mmHg, 760 mmHg, 800 mmHg, 900 mmHg, 100 mmHg, etc.) of negative pressure within the inner lumen. In some cases the elongate body further comprises an inner coil wound tube. In some cases the elongate body comprises a braided tube.

Any of these apparatuses may include other components of the system that may be coupled together, including tubing, a suction pump (vacuum), etc. In some cases the apparatus include an endoscope. Any of these apparatuses may include a control to regulate the application of suction through the overtube. For example, the control may comprise a biased valve. The control may be part of a handle (e.g., hand-held control) pedal (e.g., foot-pedal), etc. The control may be in line with the vacuum port. In some cases the control is coupled to the vacuum port and/or a fluid line (e.g., tubing) coupled to the vacuum port. For example, any of these apparatuses may include a suction tubing configured to connect the vacuum port to a source of negative pressure, and a control comprising a valve configure to control the application of suction through the inner lumen. The control may comprise a trumpet valve.

The distal end opening of the overtube may have an inner diameter that is less than the inner diameter of the inner lumen.

Any of these apparatuses may include one or more irrigators configured to pass through a lumen of an endoscope and/or the suction lumen of the overtube. For example, the one or more irrigators may comprise a radial irrigator.

In general, these apparatuses may include one or more external channels extending along a length of the elongate body. The external working channel may be expandable when so that a tool may be inserted through the working channel to expand the working channel.

Also described herein are methods of removing a material from within a body comprising: advancing an overtube and an endoscope distally within the body, wherein the endoscope is inserted through a proximal end port of the overtube into a lumen of the overtube so that a proximal end of the overtube is movably sealed around the endoscope; positioning a distal end of the overtube adjacent to a material to be removed; and applying suction through the lumen of the overtube from a port at a proximal end region of the overtube while imaging out of the distal end of the overtube from the endoscope as a user activates a control to draw the material into the overtube and around the endoscope.

Any of these method may include applying a spray of fluid from the endoscope or from a fluid line within the lumen of the overtube. Any of these methods may include coupling a suction source to the port at the proximal end region of the overtube.

Positioning the distal end of the overtube may include positioning the distal end of the endoscope adjacent to the material to be removed. These methods may include withdrawing the endoscope proximally into the overtube before applying suction through the port. The material may comprise fecal matter, blood and food debris, etc.

Any of these methods may include moving the endoscope axially within the lumen of the overtube without breaking the seal while positioning the overtube. For example, the method may include applying suction through the lumen of the overtube from the port at the proximal end region of the overtube comprises maintaining suction while the user operates the control and stopping the application of suction when the user stops operating the control. Any of these methods may include applying suction through the lumen of the overtube from the port at the proximal end region of the overtube while a user activates the control comprises applying suction while the user activates a trumpet valve. Any of these methods may include steering the distal end region of the endoscope independently of the overtube. As mentioned, these methods may optionally include rigidizing the overtube before applying suction. For example, these methods may include advancing the overtube and the endoscope comprises: rigidizing the overtube while advancing and steering the endoscope distally from the overtube, de-rigidizing the overtube, and advancing the overtube distally over the endoscope.

A method of removing a material from within a body may include: inserting an endoscope through a proximal end port of the overtube into a lumen of the overtube so that a proximal end of the overtube is movably sealed around the endoscope; positioning a distal end of the endoscope adjacent to a material to be removed; positioning a distal end of the overtube adjacent to the distal end of the distal end of endoscope and positioning the distal end of the endoscope within a distal end region of the overtube so that the endoscope images distally out of the distal end region of the overtube; and applying suction through the lumen of the overtube from a port at a proximal end region of the overtube to draw the material into the overtube and around the endoscope.

Also described herein are methods of applying fluid (e.g., washing) the body lumen. In particular, these methods may include applying radial spray (radially around the circumference of the irrigator tube) and/or applying a longitudinal spray or stream (e.g., distally from the distal end of the irrigator tube). In some cases the same irrigator tube may be configured to operate in conjunction with the endoscope and/or overtube to convert the applied spray between radial and longitudinal using just an irrigator tube that is configured to apply radial irrigation. For example, a method as described herein may include positioning an endoscope within a body region; extending an irrigator tube distally out of a lumen of the endoscope; delivering a radial spray of irrigation fluid of the irrigator tube; and withdrawing the irrigator tube proximally to deliver a longitudinal stream of irrigation fluid.

In some cases the method may include: positioning an endoscope and an overtube concentrically surrounding the endoscope within a region of a gastrointestinal (GI) tract, wherein the endoscope is within a suction lumen of a rigidizing overtube; extending an irrigator tube distally out of a lumen of the endoscope and distally out of the overtube while the overtube is held in a rigid configuration; delivering a radial spray of irrigation fluid of the irrigator tube; withdrawing the irrigator tube proximally to deliver a longitudinal stream of irrigation fluid; and applying suction through the suction lumen of the overtube to remove the irrigation fluid.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows an example of a distal end region of one example of an apparatus as described herein, including an overtube and an endoscope.

FIG. 1B is an example of the apparatus shown in FIG. 1A showing the proximal end and connection to a source of negative pressure (e.g., suction).

FIGS. 1C and 1D illustrate another example of an apparatus (e.g., system) as described herein. FIG. 1D shows an enlarged view of a portion of FIG. 1C.

FIG. 2 shows one example of an overtube, optionally configured as a rigidizing overtube as described herein.

FIG. 3A is a section through an example of a rigidizable overtube that may be rigidized by the application of negative pressure.

FIG. 3B is an enlarged view showing one example of the arrangement of layers within the elongate rigidizable overtube of FIG. 3A.

FIG. 4A is a section through an elongate rigidizable apparatus (e.g., overtube) that may be rigidized by the application of positive pressure.

FIG. 4B is an alternative sectional view showing one example of the arrangement of layers within the elongate rigidizing overtube apparatus of FIG. 4A.

FIG. 5A is an example of a proximal end of an apparatus (e.g., a suction adapter) as described herein.

FIG. 5B shows an eternal view of a suction adapter as described herein.

FIG. 5C shows a section through the suction adapter of FIG. 5B.

FIG. 5D shows an end view of the suction adapter of FIG. 5B-5C.

FIGS. 6A-6C illustrate one example of a valve (e.g., a trumpet valve) that may include as part of a control for applying suction to an apparatus as described herein. FIG. 6A shows a perspective view of the valve. FIGS. 6B-6C shows sectional views of the valve of FIG. 6A; FIG. 6B shows the valve closed (in an at-rest, unactuated state), and FIG. 6C shows the valve open (in an actuated state).

FIG. 7A-7B show an example of an adapter, configured as a threaded adapter. FIG. 7A is side view, and FIG. 7B shows a section through the adapter of FIG. 7A.

FIG. 8A shows an exploded view of a portion of an adapter (proximal end) similar to that shown in FIG. 7A-7B.

FIG. 9 is an example of an integrated rigidizing overtube including a proximal endoscope seal and/or a suction port.

FIGS. 10A-10E illustrates one example of a method of operating an apparatus as described herein.

FIGS. 11A-11F illustrate one example of a method of using an apparatus as described herein.

FIG. 12A schematically illustrates an example of an apparatus as described herein including irrigation through the endoscope.

FIG. 12B schematically illustrates an example of an apparatus as described herein including irrigation thought a separate irrigation line.

FIGS. 13A-13B schematically illustrate another variation of an apparatus as described herein; in this example the vacuum exit is integrated into the handle.

FIGS. 14A-14C illustrate another example of a suction adapter for use with (or to be integrated into) an overtube as described herein. In FIGS. 14A-14C the adapter includes a swivel valve, shown in different orientations.

FIGS. 15A-15C illustrate examples of a control (e.g., a lever valve) for controlling the application of suction as described herein. FIG. 15A-15B illustrate a first example of a lever valve in a side and side perspective views, respectively. FIG. 15C shows another example of a lever valve in section view.

FIGS. 16A and 16B show an example of a control (configured as a lever valve) for controlling the application of suction as described herein. FIG. 16A shows a side view and FIG. 16B shows a sectional view through the lever of FIG. 16A.

FIG. 17A-17C illustrate one example of a control (configured as a foot petal) for controlling the application of suction. FIG. 17A shows a front view, FIG. 17B shows a side perspective view and FIG. 17C shows a top perspective view.

FIGS. 18A-18B illustrate the bending flexibility of a different examples of an overtubes. FIG. 18A shows an overtube with a low bending flexibility and FIG. 18B shows an example of an overtube as described herein, having a high bending flexibility and a high hoop strength.

FIG. 19 illustrates an example of a distal end region of an overtube apparatus having a plurality of suction relief holes.

FIG. 20 illustrates an example of a robotic system including an apparatus as described herein.

FIGS. 21A-21B illustrate an example of releasing suction against a wall of a body lumen using an air relive valve.

FIGS. 21C-21D illustrate an example of applying a fluid through the suction lumen of the overtube to remove a clog.

FIGS. 22A-22C illustrate examples of an overtube including rinsing channels. FIG. 22A shows an example of an overtube from which an endoscope is extending, the overtube having a plurality of rinse channels flush with the distal end of the overtube. FIGS. 22B-22C show side perspective and side views, respectively, of an overtube from which an endoscope is extending, with a plurality of angled rinse channels extending distally at an angle relative to the distal end of the overtube.

FIG. 23A shows an example of an irrigator tube providing a radial spray.

FIG. 23B shows an example of an irrigator tube providing a longitudinal spray (e.g., stream).

FIGS. 24A-24B schematically illustrate an example of an apparatus including an irrigator tube configured to apply a radial spray. FIG. 24A schematically illustrates a section through an apparatus showing deployment of the irrigator tube distally out of a lumen of the endoscope housed within an overtube. FIG. 24B shows the irrigator tube extending distally out of the endoscope and out of the overtube, to provide a radial spray into the body lumen.

FIGS. 24C and 24D illustrate conversion of the applied spray from the radial irrigator tube of FIGS. 24A-24B into a linear stream. In FIG. 24C the radial spray is converted into a distal linear spray by applying the spray from the irrigator tube within the distal end region of the overtube but external to the endoscope. In FIG. 24D the radial spray is converted into a distal linear spray by applying the spray from the irrigator tube within the lumen of the endoscope.

FIGS. 25A-25J illustrate examples of different nozzle types that may be used at the distal end of an irrigator tube to achieve different spray/stream patterns.

FIGS. 26A-26H illustrate examples of different nozzle types that may be used at the distal end of an irrigator tube to achieve different spray/stream patterns.

FIGS. 27A-27C illustrate an example of an apparatus (e.g., including an overtube) having one or more external working channels arranged along the outer length of the overtube.

FIG. 28A shows an examples of a suction or aspiration tube that may be used with any of the apparatuses described herein.

### DETAILED DESCRIPTION

Described herein are methods of removing material from a patient's body, including methods of removing debris (e.g., fecal matter), blood, clot material, etc., from within a body lumen, such as the GI tract. In some cases, these methods may be used for cleaning material from a colon or other body lumen. These methods may be used as part of another procedure, such as for assisting in imaging and/or surgical procedures to treat a colon, e.g., as part of a colonoscopy.

These apparatuses (e.g., device, systems, etc.), may include an overtube, which may be referred to herein as a suction overtube, that may be configured to operate in conjunction with an endoscope. The overtube may have an elongate body having an inner lumen that extends from a distal end to a proximal end. The overtube may be configured to receive and seal an endoscope within the inner lumen and may have a proximal end region that includes an endoscope receiving port that is configured to receive an endoscope therethrough into the inner lumen. The endoscope receiving port may be in-line with the inner lumen of the elongate body and is configured to seal around endoscope to allow distal/proximal movement of the endoscope relative to the overtube, without substantial loss of suction within the inner lumen. In general, the overtube may also include a vacuum port at the proximal end region, distal to the seal for the endoscope at the distal end region of the overtube, that is in fluid communication with the inner lumen, and through which negative pressure may be applied. The overtube may be configured to withstand the application of negative pressure within the inner lumen while still allowing movement of the endoscope within the inner lumen.

In any of these apparatuses, the elongate body may be rigidizing (e.g. a rigidizing overtube). Thus, the overtube may be configured to convert between a flexible configuration and a more rigid configuration by the application or release of pressure within a wall of the elongate body. The pressure applied to convert between the rigid configuration and the less rigid configuration (e.g., becoming more rigid with the application of pressure) may be either positive pressure or in some cases, negative pressure. Thus, a rigidizing overtube may include a rigidizer port (e.g., pressure port) that is configured to receive positive and/or negative pressure to convert the elongate body from a flexible to a rigid configuration. The rigidizer port may be at the proximal end region.

In some cases the overtube may be integrated with the vacuum port and the endoscope receiving port. Alternatively, in some examples the overtube may be separate from and may couple to an adapter that includes the vacuum port and the endoscope receiving port.

Thus, in some cases the methods and apparatuses described herein include an overtube including an inner lumen that is configured to receive both an endoscope and receive suction in order to remove material from within a body, without requiring removal of the endoscope. In general, the overtube apparatuses may include an elongate body having an inner lumen that extends from a distal end to approximal end of the overtube. The apparatus may also include a proximal end region configured to receive an endoscope so that the endoscope may move within the inner lumen of the endoscope, e.g., distally and proximally. The receiving port may be at the proximal end so and arranged so that the endoscope may be inserted into the receiving port and through the receiving port into the inner lumen. These apparatuses may also include a vacuum port at the proximal end region that is in fluid communication with the inner lumen. In general, the apparatus may be configured so the user (e.g., doctor, surgeon, nurse, technician, etc.) may activate and maintain the suction through the inner lumen, and may stop applying suction (e.g., when the control is no longer being activated by the user).

The apparatuses described herein may be configured for use in one or more of: the neurovasculature (e.g., aortic arch, subclavian, carotid, vertebral, basilar, posterior cerebral, circle of Willis, middle cerebral, anterior cerebral, etc.), the upper GI tract (mouth esophagus, stomach, pylorus, bile duct and pancreatic duct, etc.), the small bowel (e.g., small intestine, duodenum, jejunum, ilium, etc.), the lower GI tract (rectum, regions of colon, e.g., sigmoid, descending, transverse, ascending, cecum, ileocecal valve, etc.), the urinary tract (urethra, bladder, kidneys, ureters, etc.), the peripheral vasculature (e.g., femoral, iliac, mesenteric, lumbar, renal, celiac trunk, hepatic, thoracic, etc.), the cardiac region (e.g., aorta, right coronary artery, left coronary artery, etc.), the left heart (e.g., aorta, aortic valve, left ventricle, etc.), the right heart (e.g., vena cava, right atrium, left atrium, mitral valve, coronary sinus, tricuspid valve, right ventricle, pulmonary valve, pulmonary vasculature, etc.) and/or the right pulmonary region (e.g., mouth, larynx, trachea, bronchial tree and lobes etc.).

The apparatuses described herein may be configured as integrated overtubes that include the proximal end region configured to receive an endoscope and/or a suction port (e.g., vacuum port) for applying suction through the inner lumen of the overtube, or they may be configured to convert an existing overtube (see, e.g., U.S. Pat. No. 11,135,398, titled “DYNAMICALLY RIGIDIZING COMPOSITE MEDICAL STRUCTURES”, herein incorporated by reference in its entirety). For example, described herein are adapters that are configured to couple to the distal end of an existing overtube to convert it to allow operation of the overtube as described herein. In some cases, the overtube may optionally be a rigidizing overtube; however these methods and apparatuses may be non-rigidizing overtubes.

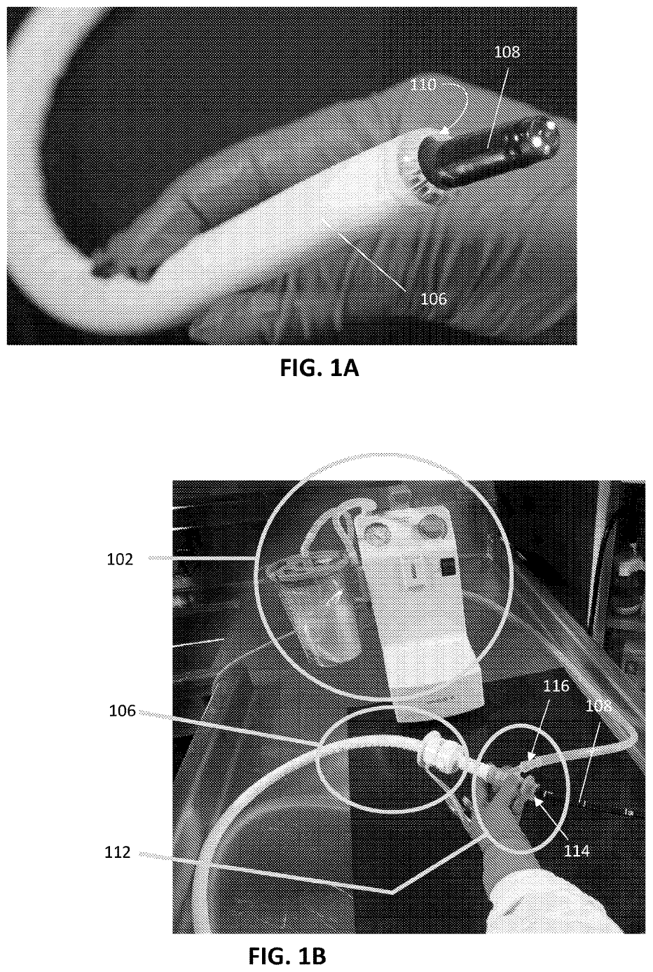

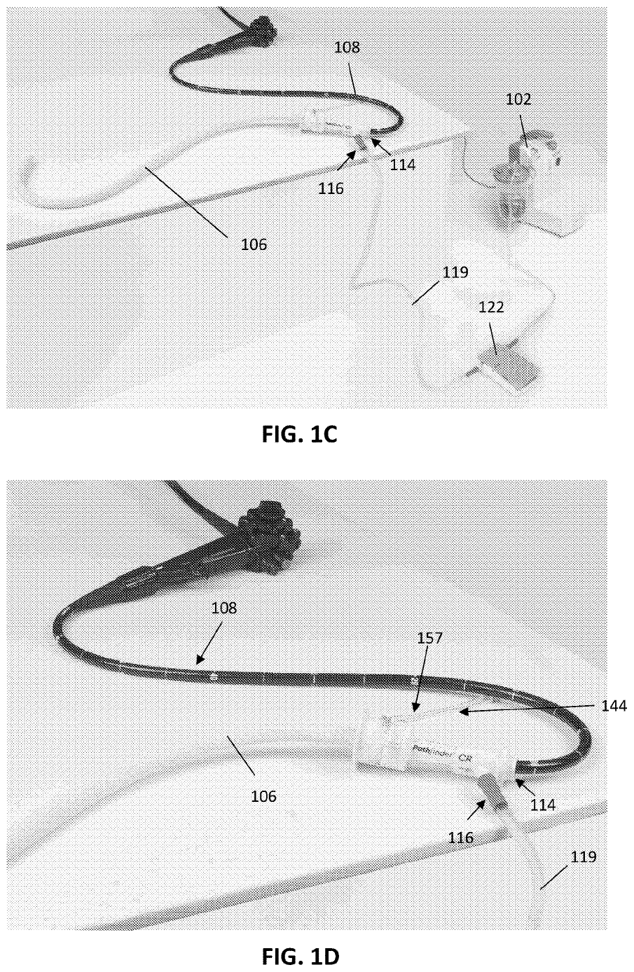

FIG. 1A illustrates an example of a distal end region of a flexible overtube 106 having a distal end region 110 out of which suction may be applied. In this example, the overtube is a rigidizing overtube. An endoscope 108 is shown extending distally from the overtube. FIG. 1B shows an example of a system including the overtube 106, showing the proximal end region of the overtube including an adapter 112 (e.g., suction adapter) coupled to the proximal end of the overtube. The suction adapter portion of the apparatus includes a proximal end region including an endoscope receiving port 114 configured to receive an endoscope 108 therethrough into the inner lumen. The endoscope receiving port is in-line with the inner lumen of the elongate body. The suction adapter portion of the apparatus also includes a vacuum port 116 that is in fluid communication with the inner lumen and is shown connected to a vacuum source 102. In general, the vacuum port may be connected to a control (not shown in FIG. 1B) for controlling suction through the overtube. In some examples the control includes a valve or clamp that may be closed at rest, but may be held open by a user applying actuation (e.g., pushing, squeezing, gripping, depressing, etc.) to maintain the valve open (fully open or selectively opened more or less). The valve may be manually operated, or it may be electronically operated.

In the example shown in FIGS. 1A and 1B, the overtube is configured as a medium-sized overtube (e.g., having an inner diameter of 14 mm) and the endoscope shown as a gastroscope (having a tip diameter of approximately 9 mm). Any appropriate size overtube and endoscope may be used. The apparatus may also include a control coupled to the vacuum port and configured to apply suction through the vacuum port when the control is held actuated by a user (not shown in FIGS. 1A-1B).

FIGS. 1C and 1D show another example of an apparatus (e.g., system) including an overtube 106 similar to that shown in FIGS. 1A-1B, for use with an endoscope 108. The endoscope may be provided with the apparatus or may be separately supplied, e.g., as a commercial endoscope. The apparatus shown in FIG. 1C also includes a source of vacuum 102, and tubing connecting the vacuum to a vacuum port 116 on the proximal end of the overtube. A controller 122 is connected to this tubing to turn suction on/off through the overtube inner lumen. The controller is shown as a foot petal in this example. The controller may include one or more valves (e.g. pinch vales) regulate the applied suction. The overtube proximal end also include an endoscope receiving port 114 configured to receive an endoscope 108 therethrough into the inner lumen. The endoscope receiving port is in-line with the inner lumen of the elongate body. The vacuum port 116 is in fluid communication with the inner lumen and is shown connected to the vacuum source 102 by tubing 119. In this example the overtube also includes a rigidizer port including a pressure line 144 that is configured to receive positive and/or negative pressure to convert the elongate body from a flexible to a rigid configuration. A separate irrigation/flush line 157 is also shown in this example, which may provide fluid (e.g., water) into the inner lumen. The apparatus may also include a separate source of pressure (positive and/or negative pressure) for rigidizing the overtube. In some cases the same vacuum source 102 (or a duplicate source) may be used.

In general, the overtubes described herein may be configured to withstand the application of suction, without collapsing. Thus, in general, these apparatuses may have a hoop strength that is capable of withstanding up to, e.g., up to 760 mm Hg (one atmosphere) of negative pressure or more, without collapsing. Further, these apparatuses may be configured to maintain a very high degree of flexibility (when in a flexible configuration). For example, in some variations the overtube includes one or more reinforcing layers including reinforcing coils and/or braided layers (e.g., braided shafts). Any of these apparatuses may include a coil-wound reinforcing tube. For example, a reinforced outer layer may be reinforced with a plurality of filaments wound around the layer (or within the layer). An inner tube (e.g., inner coil-wound tube, ICWT) may include one or more reinforcing wires. The inner coil-wound tube may be adjacent to other layers. In some examples the overall bending flexibility may be increased, even with the requirement to withstand high pressures, by using low durometer materials. For example, the overtube body may be formed of a material having a durometer that is less than 90 Shore A, and in particular less than 80 Shore A (or less than 75 Shore A, less than 70 Shore A, less than 65 Shore A, less than 60 Shore A, less than 55 Shore A, less than 50 Shore A, etc.). For example, the body of the overtube may be made of one or more layers of such low durometer material, and may be reinforced with a reinforcing layer, such as (in some examples) a helically wound coil reinforcing layer.

Any of these apparatuses may also include torsional stiffening elements (e.g., a torsional stiffening layer). The torsional stiffening layer may be integrated into the inner and/or outer layers. Alternatively, it may be free-floating such that is not intentionally attached to adjacent layers.

The overtubes described herein may be any appropriate length, but in particular may be long, and may be adapted for use with long scopes (e.g., 50 cm or longer, 55 cm or longer, 60 cm or longer, 65 cm or longer, etc.). Shorter or longer lengths may also be included, which may be useful for certain anatomical targets.

In addition, the overtubes described herein may be configured to a distal tip region that is adapted for use with the endoscope and applying suction. For example, the distal end region of the overtube may include an opening into the lumen of the overtube through which suction may be applied and through which the endoscope may be positioned. The distal opening into the overtube may be configured to allow the endoscope to be extended out of and retracted into the overtube. In some examples the distal opening may be narrowed compared to the more proximal inner diameter of the lumen of the overtube. This narrowing may help center, support and/or guide the endoscope. The narrowing may be, e.g., between 98% and 50% of the inner diameter of the more proximal region of the inner lumen of the overtube (e.g., between about 98% and 55%, between about 98% and 60%, between about 98% and 65%, between about 98% and 70%, between about 98% and 75%, between about 98% and 80%, etc.).

The distal tip region, including the distal end opening, of the overtube may be transparent in order to allow visualization through the distal end, e.g., by the endoscope when retracted into the lumen of the overtube. As mentioned above, any of the apparatuses described herein may include vacuum release openings (e.g., holes or openings at the distal end and/or proximal to the distal end to allow passive venting to prevent suction locking onto the tissue (e.g., wall of the body, etc.).

Any of the overtubes described herein may be steerable, particularly at the distal end region (e.g., the distal end region of the overtube). The overtube may be steerable by mechanical steering (e.g., pullwires, tendons, shape memory alloy, etc.), pneumatic (pressure-driven steering), electrical/magnetic steering, etc.

Optionally, any of the apparatuses described herein may include an overtube and/or endoscope that is rigidizable. For example, an overtube may be controllably converted between a flexible configuration and a rigid (less flexible) configuration. These apparatuses can transition quickly from a flexible configuration (i.e., one that is relaxed, limp, or floppy) to a rigid configuration (i.e., one that is stiff and/or holds the shape it is in when it is rigidized). The rigidizing apparatus (e.g., overtube) may include a plurality of layers (e.g., coiled or reinforced layers, slip layers, rigidizing layers, bladder layers and/or sealing sheaths) can together form the wall of the rigidizing apparatuses, which may be referred to as “layered rigidizing apparatuses.” The rigidizing apparatuses described herein may be rigidized by jamming particles, by phase change, by interlocking components (e.g., cables with discs or cones, etc.) or any other rigidizing mechanism. The rigidizing apparatuses can transition from the flexible configuration to the rigid configuration, for example, by applying a vacuum or pressure to the wall of the rigidizing apparatus or within the wall of the rigidizing apparatus. With the vacuum or pressure removed, the layers can easily shear or move relative to each other. With the vacuum or pressure applied, the layers can transition to a condition in which they exhibit substantially enhanced ability to resist shear, movement, bending, torque and buckling, thereby providing rigidization.

Any of the rigidizable apparatuses described herein may include rigidizing layers or regions that engage with a compression layer (which may be or may include a bladder) that applies force to the rigidizing layer to rigidize the rigidizing layer or in some cases to de-rigidize (e.g., release from rigidization) the rigidizing layer. In some examples, these rigidizable apparatuses may include a rigidizing layer that could include a braid, knit, woven, chopped segments, randomly distributed or randomly oriented filaments or strands, engagers, links, scales, plates, segments, particles, granules, crossing filaments, or other materials forming the rigidizing layer. For example, the rigidizing layer may comprise multiple strand lengths or strand segments that cross over each other (e.g., as part of a braid, knit, woven, etc.); the compression layer may apply force to drive the crossing strand lengths or strand segments against each other. Although many of the examples shown herein are braids, any of these apparatuses may instead or in addition include a general rigidizing layer comprising crossing strand lengths or strand segments.

The examples of rigidizing apparatuses described herein may use pressure (positive pressure) and/or negative pressure to selectively and controllable rigidize. In some examples the method described herein may be used with any appropriate rigidizing apparatus.

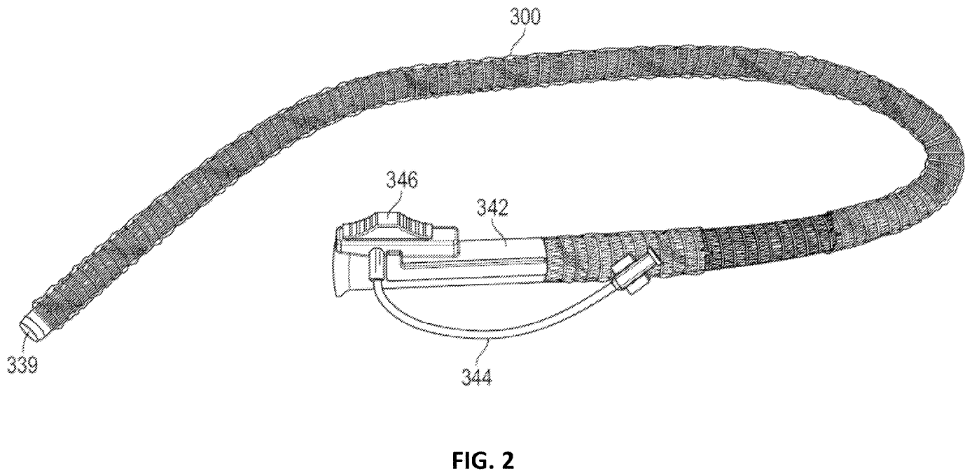

An exemplary rigidizing overtube apparatus is shown in FIG. 2. The apparatus shown includes a rigidizing apparatus 300 having a wall with a plurality of layers including a rigidizing layer, an outer layer (part of which is cut away in this example to show the rigidizing layer thereunder, configured as a braid layer in this example), and an inner layer.

The system further includes a handle 342 having a vacuum or pressure inlet 344 to supply vacuum or pressure to the rigidizing apparatus 300. An actuation element 346 can be used to turn the vacuum or pressure on and off to thereby transition the rigidizing apparatus 300 between flexible and rigid configurations. The distal tip 339 of the rigidizing apparatus 300 can be smooth, flexible, and atraumatic to facilitate distal movement of the rigidizing apparatus 300 through the body. As mentioned above, the tip may be adapted to be narrowing, transparent, etc.

In addition to tapered radially inward so that the inner lumen opening has a narrower open diameter than the inner diameter of the inner lumen mentioned above, in any of these examples the outer diameter of the distal tip may be tapered to be atraumatic. For example, the tip 339 can taper from the distal end to the proximal end to further facilitate distal movement of the rigidizing apparatus 300 through the body. In this example, the rigidizing apparatus is configured as an overtube, but other configurations may be used.

The rigidizable apparatuses and methods described herein may be part of a medical access system for treating regions of the body that are otherwise hard to access and operate within, particular during minimally or non-invasive procedures.

Rigidizing apparatuses as described herein may be configured to rigidize when negative pressure and/or positive pressure is applied. These rigidizing apparatuses as described herein may be used in conjunction with other rigidizing apparatuses that rigidize with other methods, including those that do not rely upon the application of positive or negative pressure. For example, a rigidizing apparatus may be configured to include multiple layers arranged into an elongate catheter-like body. The proximal end of the device may include a handle or other manipulator and may include a connection to one or more pressure sources. Applying pressure from the pressure source may be controlled by multiple methods, including operation of a handle or an electronically controlled device. Control may result in a pressure differential that causes the device to transition between a highly flexible configuration, allowing the tubular body to readily bend, when steered or otherwise guided (e.g., over a guidewire, etc.), and one or more (e.g., a continuum) of rigid configurations. In some examples, particularly (but not exclusively) in reference to apparatuses that rigidize based on the application of positive pressure, the rigidity of the elongate body is proportional to the applied pressure differential, so that the greater the pressure differential, the more rigid the device may become over at least a range of pressure differential values.

As mentioned, these apparatuses may include multiple layers (which may be concentrically arranged around the inner lumen), including a rigidizing layer and at least one of an outer or inner layer. Many of these examples also include a compression layer that may engage with the rigidizing layer, and in some examples the apparatus may include a combined rigidizing layer/compression layer. Described herein are rigidizing layers that may be particularly well suited to rapid and precise actuation over a variety of pressures, including in particular positive pressures (e.g., high positive pressures, i.e., atm of about 2 or more, 4 or more, 6 or more, 8 or more, 10 or more, 15 or more, 20 or more, 30 or more, etc.). Any of these apparatuses may also be configured so that at least some of the inner and/or outer layers making up the rigidizable device have different durometers on the inner and outer portion of either the inner or outer layers. Also described herein are apparatuses and methods including nested sets of rigidizable apparatuses, which may include any of these rigidizable devices. Any of these apparatuses may include one or more torsional enhancing layers for improving torsional control, particularly when included as part of a nested pair of rigidizable devices (e.g., as part of the inner, or child, device).

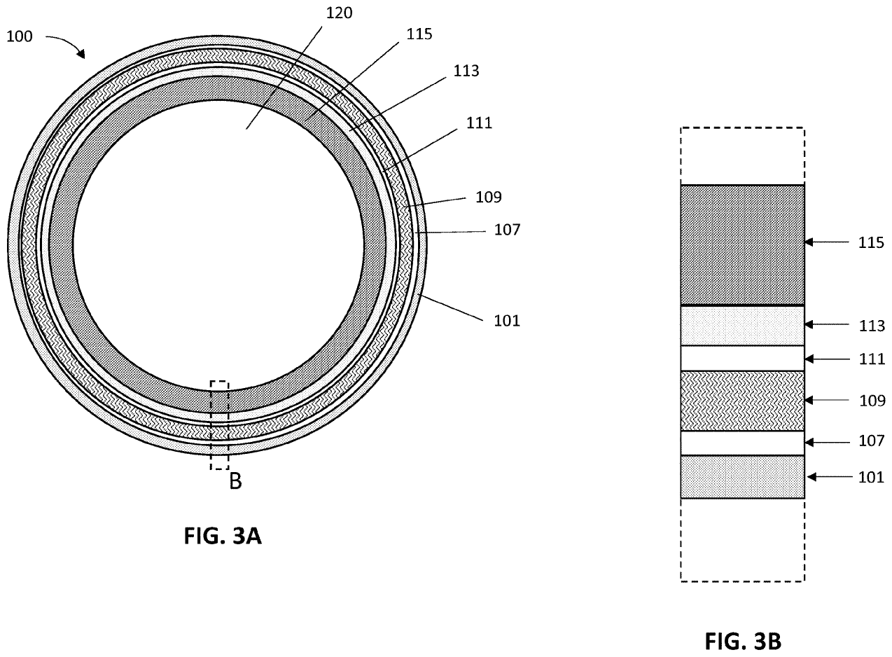

FIG. 3A illustrates an example of a transverse section through an elongate rigidizing apparatus, showing the arrangements of the many layers that may be included. In this example the rigidizable device 100 is configured to be actuated by the application of a negative pressure (e.g., vacuum). The device 100 shown includes an inner layer 115 that may be reinforced (e.g., by including one or more reinforming members, such as a helically arranged strip, ribbon or wire), an optional slip layer 113, a gap 111, a rigidizing layer 109, configured in this example as a braid layer, a second gap 107 and an outer layer 101. The layers surround a central lumen 120. In some examples a vacuum may be applied between the outer layer and the inner layer to rigidize. For example, a port configured to couple to the source of negative pressure may be located at the proximal end of the device and may be in fluid communication with the gap region 107 between the flexible outer layer 101 and the rigidizing layer 109, e.g., braided layer. Thus, in this example the outer layer may act as a compression layer. FIG. 3B shows a section through one wall region B of the cylindrical-shaped body of the device. Applying suction may allow the outer layer 101 to be drawn onto the rigidizing layer, causing it to rigidize, limiting or preventing bending of the device.

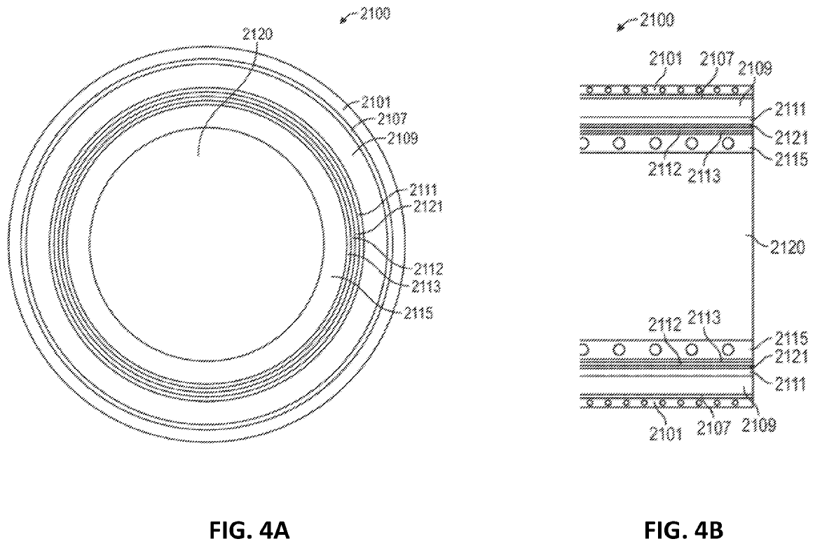

Another example of a rigidizable apparatus, e.g., rigidizing overtube 2100 having an inner lumen 2120, is shown in FIGS. 4A-4B. In this example the device may also be an elongate, e.g., catheter or tubular-shaped device similar to that in FIGS. 3A-3B but may be rigidized by the application of positive pressure. For example, FIG. 4A shows a section transverse to the long axis of an elongate rigidizable device. In this example, the layers forming the device are arranged so that an inner reinforced layer 2115 is the most radially-inward layer and may be reinforced, e.g., by a helically wound ribbon, strip, cable, etc. The device may also include an optional slip layer 2113 which may reduce the friction between the inner layer and the more radially-outward layers. The slip layer may be a powder, or it may be a lubricious layer or a layer of lubricious material. A first gap 2112 layer is shown separating the inner layer 2115 and/or the slip layer 2113 from a compression layer, configured in this example as a bladder layer 2121. A second (or intermediate) gap layer 2111 spaces the bladder layer from the rigidizing layer 2109, shown in this example as a braid layer. A third gap layer 2107 is positioned between the rigidizing layer and an outer layer 2101. The outer layer in this example (similar to the inner layer 2115) is reinforced, for example, by a helically wound filament, wire, fiber, band, etc. Although not shown, when actuated by the application of positive pressure between the compression (e.g., bladder) layer and the inner layer, the bladder layer may push the braid layer into the outer layer to rigidize the rigidizing layer.

Both examples of a devices shown in FIGS. 3A-3B and 4A-4B may include additional optional layers or components. Further, the compositions of the rigidizing layers may be modified in order to improve performance. In particular the rigidizing layer may be modified to include structures (e.g., knits, woven, braids, scales, plates, arrays of filaments, granules, and combinations thereof, etc.) that may enhance or improve performance. Rigidizing elements may be used as one type alone, or in conjunction with other rigidizing elements. In some examples the inner and/or outer layers may be modified to enhance or improve performance, including the addition of torsional control components, and/or modulating the durometer of the inner and outer regions of these layers.

As mentioned above, in addition to (or instead of) the overtube, the endoscope may be rigidizing. In general, the apparatuses described herein are configured to apply suction through the overtube, and around an endoscope within the overtube. In some examples, suction may also be applied through the endoscope. The endoscope may be applied to the same source or suction, or to a different source of suction. In some examples the apparatus may be used by applying suction first to the endoscope, e.g., to coordinate moving material to be removed into or near the mouth of the overtube, then applying suction through the overtube to remove material. Thus, the combination of suction from the endoscope and suction from the overtube may be coordinated to remove material. In some cases, the use may limit the use of the endoscope to bringing material to the opening of the overtube, in order to prevent clogging of the endoscope. Any of these methods may alternatively or additionally include a dedicated pressure release line and/or a pressure release line for each of the overtube and the endoscope.

In some examples the apparatus may be configured to apply positive pressure through the endoscope, e.g., by applying pressurized fluid through the suction lumen or working channel of the endoscope, particularly while the endoscope distal tip is within the lumen of the overtube. This may allow the apparatus to clear a clog within the lumen of the endoscope without risking the release of material within the body. Any of these apparatuses may also or additionally include one or more fluid lines, which may be inserted into the inner lumen of the overtube separately from (or in some cases in addition to and/or through) the endoscope. Thus, fluid line may apply fluid (e.g., water) into the lumen of the overtube and/or out of the distal end of the overtube. Fluid may be applied as a spray or stream from a nozzle on the distal end region and/or tip of the apparatus. In some examples fluid may be applied through an irrigation line within the overtube to clear clogs near the proximal end of the overtube.

In some examples, by pressurized fluid may be applied through the suction lumen or working channel of the endoscope, a stream of high pressure fluid can be used to break apart material, including clot, food, or feces.

The apparatuses described herein may generally form a seal around the endoscope, e.g., in the region of the overtube (and in some examples in the region of the suction adapter) to maintain a seal around the endoscope so that suction may be applied through the inner lumen of the overtube without substantial loss of suction from the proximal end, where the endoscope inserts into the overtube lumen.

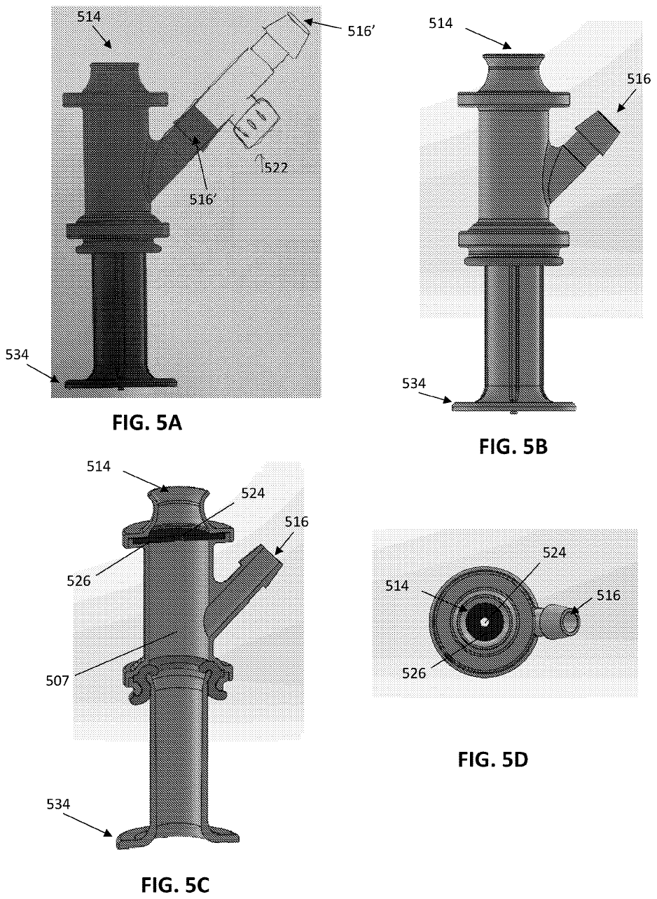

FIG. 5A illustrates one example of the proximal end of an apparatus, which may be integrated with the overtube, or it may be configured as a removable adapter (also referred to herein as a suction adapter) for coupling with the overtube (e.g., at coupling region 534), or attached via a flexible vacuum line. In FIG. 5A the proximal end region of the apparatus (e.g., suction adapter) includes an endoscope receiving port 514 that is configured to receive an endoscope. The apparatus also includes a vacuum port 516 configured to couple with a vacuum source and to apply suction within the inner lumen of the overtube. A control 522 may be included in line with the overtube inner lumen and the vacuum port 516. In general the control 522 may be configured to be controlled by a user to control the application of suction through the overtube. The control may be coupled to and/or integrated into the overtube and/or suction adapter, as shown in FIG. 5A, or it may be separate, and connected to the source of suction (negative pressure) via a control suction port 516′ and/or between the source of negative pressure and the vacuum port 516 of the adapter region.

FIGS. 5B-5D illustrate examples of a suction adapter portion of an apparatus that may be configured as a separate adapter for use with an overtube, or may be integrated with the overtube. FIGS. 5B-5C show side views of the adapter region, similar to that shown in FIG. 5A, but without the attached or integrated control (including the valve) for controlling the application of suction through the apparatus. As shown in the sectional view of FIG. 5C, the endoscope receiving port 514 includes a seal 526. In this example the seal 526 is configured as a sheet of material (e.g., an elastomeric material) having seal opening 524 therethrough. The seal opening has a smaller diameter than the outer diameter of the endoscope to be inserted into the overtube lumen 507. The seal may be lubricated or may be used with lubrication, so that the endoscope may slide within the seal while still maintaining the seal to prevent the loss of vacuum when suction is applied. FIG. D shows a top view of the suction adapter of FIG. 5B-5C, showing the seal opening 524 of the seal 526, visible through the endoscope receiving port 514. The seal may be coated to enhance lubricity and/or easy of sliding (e.g., by a hydrophilic coating or other lubricious coating).

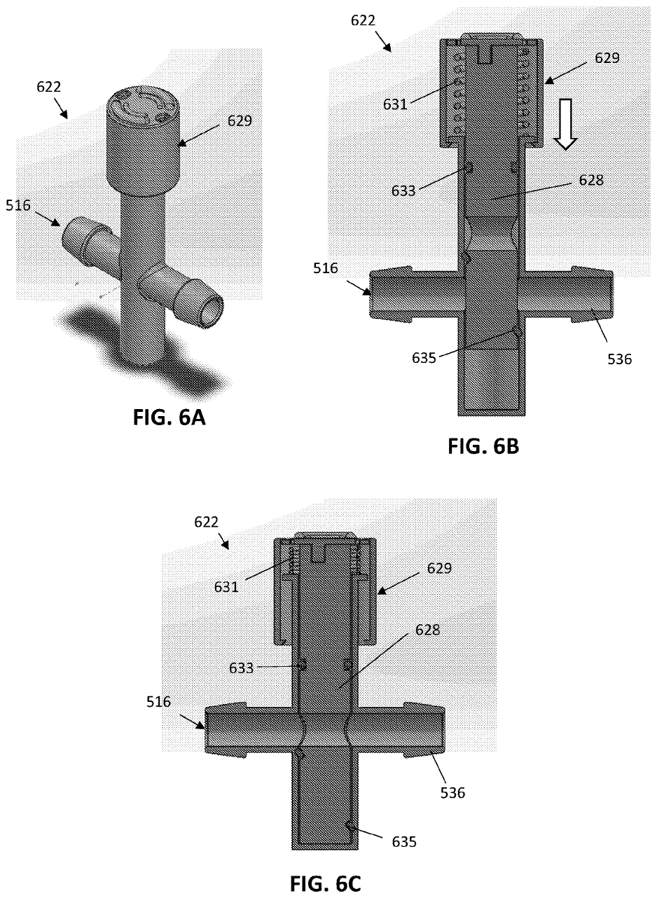

In FIG. 5A the control includes a valve that is biased by a bias (e.g., spring) that is configured to hold the valve in the closed position unless the user applies force to displace the spring, as illustrated in the example shown in FIGS. 6A-6C, described below. In some configurations the control may include a spring-loaded trumpet valve 622, an example of which is shown in FIGS. 6A-6C. FIG. 6A shows an external illustration, while FIGS. 6B and 6C shows a section through the trumpet valve of FIG. 6A in an open (FIG. 6B) and closed (FIG. 6C) configuration. The valve in this example includes a vacuum port 516 configured to couple to a source of vacuum and an outlet 536 to couple to the lumen of the overtube. The spring-loaded valve mechanism includes a shaft 628 that may be displaced down to open a connection between the vacuum port (and therefore a connected source of vacuum) and the suction lumen outlet 536, when the control input 629 (e.g., button, knob, etc.) is driven down, e.g., by the application of force from the user. For example, the user may push down on the control to compress a spring 631 within the valve to displace the shaft 628, forming a connection allowing suction to flow from the suction port and out of the distal end of the suction lumen of the overtube. In FIG. 6B the control 622 with the valve is shown in the closed (at rest) configuration the shaft 628 includes a pair of seals 633, 635 configured to prevent leakage from between the vacuum port 516 and the suction lumen outlet 536. In FIG. 6C the valve of the control is shown in an open (actuated) configuration, e.g., when the actuator (control input 629) is held down, compressing the bias (spring 631) and displacing the shaft 628 within the valve to form a connection between the suction lumen outlet 536 and the vacuum port 516.

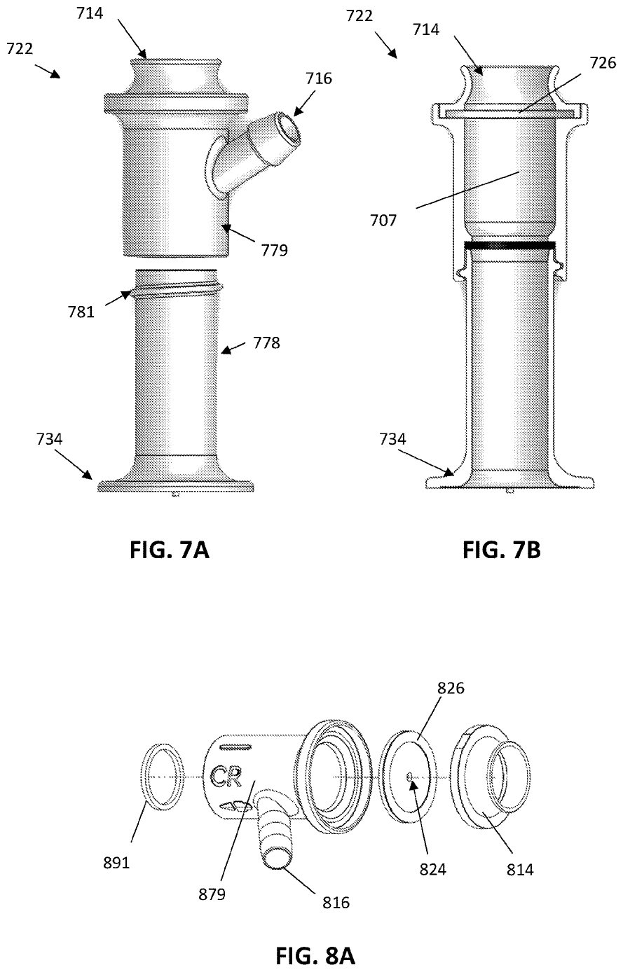

FIGS. 7A-7B illustrate another example of an adapter 722 for attaching to a proximal end of an overtube to convert it into an apparatus for sealingly receiving an endoscope and applying suction through the overtube. In FIG. 7A the example shown is configured to include two parts, a proximal portion 779 and a distal portion 778; the two are configured to mate together. In this example, the distal portion 778, which may be coupled to or integral with the elongate body of the overtube (e.g. via a coupling region 734), is configured to have a threaded engagement 781 that mates with a channel or complementary region on the distal portion. The distal portion also includes the endoscope receiving port 714 that is configured to receive an endoscope and a vacuum port 716 configured to couple with a vacuum source (and/or controller) and to apply suction within the inner lumen of the overtube. A separate controller (e.g. foot petal, hand control, etc.) that may valve the connection (e.g., tubing) between the suction port and the source of suction may be included (not shown).

FIG. 7B shows a section through the assembled adapter 722 of FIG. 7A. In this example, the internal seal 726 moving sealing an endoscope in communication with the inner lumen 707 of the overtube is shown. The seal may include one or more elastomeric layers, rings (e.g., o-rings) or the like that may allow insertion and removal of the endoscope while preventing or reducing leaking that would disrupt the application of suction through the inner lumen of the overtube. As mentioned, the seal may include an opening.

FIG. 8A shows an exploded view of the distal region 879 of an adapter similar to that shown in FIGS. 7A-7B. In this example, the proximal portion 779 includes the suction port 816 and a thread-receiving region 891 within the inside of the proximal region. The proximal end of this portion of the adapter the endoscope seal 826, which is shown as an elastomeric layer having an opening 824 for receiving the endoscope. The seal may be reinforced, e.g., radially reinforced near the wall. The proximal end may be configured as the endoscope receiving port 814.

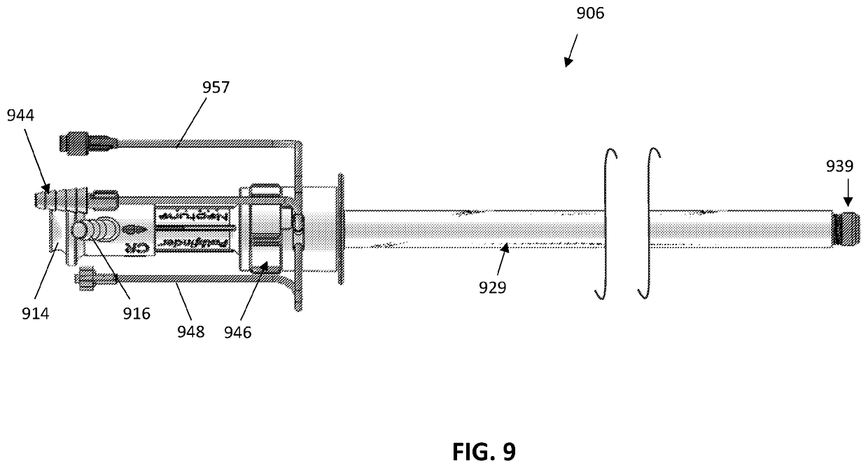

As mentioned above, any of these apparatuses may be integrated so that the overtube is integrated to include the endoscope receiving port and suction port. FIG. 9 illustrates one example of an integrated apparatus configured as an overtube that includes an endoscope receiving port 914 at the proximal end, which includes a seal (not visible) as described above. the proximal end also includes a vacuum port 916 which is configured to fluidically couple with a suction line connected to a source of suction (and in some cases, a controller). The overtube 906 in this example also includes an elongate body 929 that may be configured to rigidize, e.g., to controllably and reversibly transition between a relatively flexible configuration and a more rigid (e.g., less flexible) configuration by the application of pressure (positive and/or negative pressure). In FIG. 9, the apparatus includes a rigidizing port 944 that may include a length of connector tubing for coupling to a source of negative or positive pressure. In some cases the overtube may include an actuator 946 (e.g., switch, dial, button, etc.) on the proximal end (e.g., handle region) of the overtube itself or separately, e.g. between the rigidizing port and the internal layers of the elongate body of the overtube that may be rigidized. The example shown in FIG. 9 also include a flush line 957 and an optional pressure release line 948 (e.g., air release line). The distal end 939 of the apparatus shown in FIG. 9 is tapered radially inward, which may help center the endoscope that may extend distally out of the overtube and may also enhance suction into the overtube.

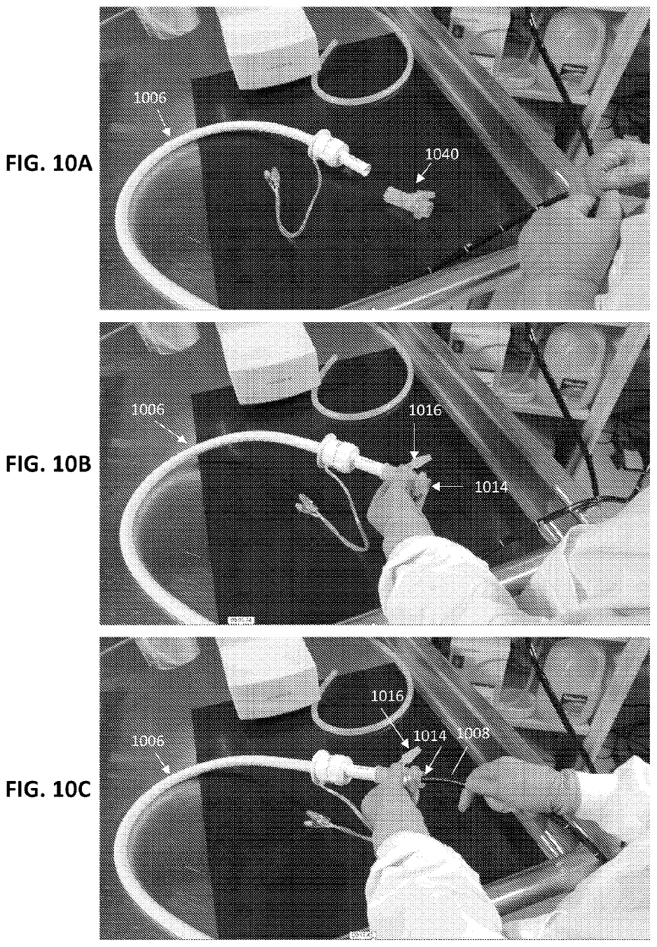

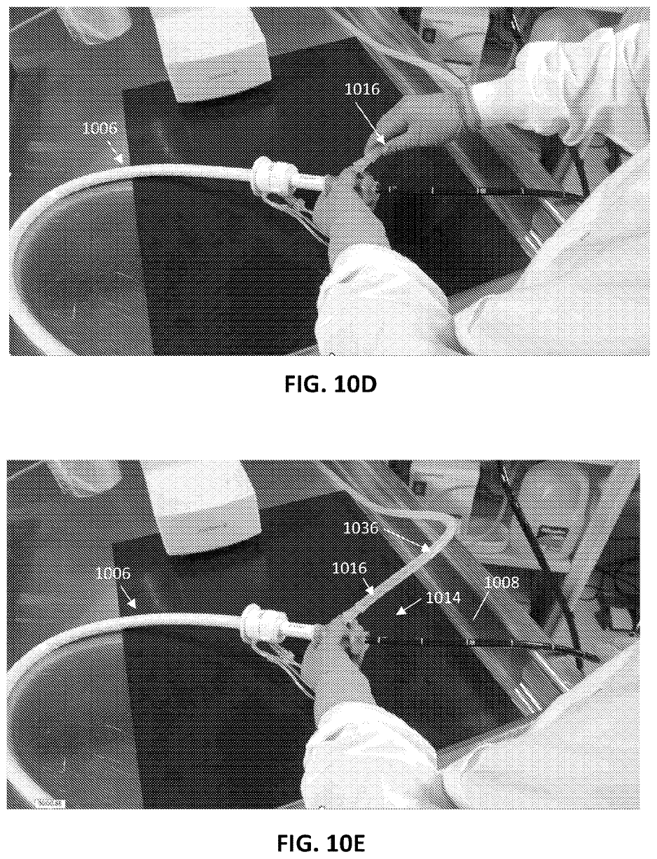

FIGS. 10A-10E illustrate the preparation of an apparatus including an overtube as described herein. FIG. 10A shows the overtube 1006 in an elongate and flexible configuration. The suction adapter 1040 portion of the apparatus is shown adjacent, but not yet connected, to the proximal end of the overtube. In FIG. 10B the suction adapter portion is coupled to the proximal base of the overtube. The suction adapter 1040 includes a vacuum port 1016 configured to couple to a source of suction, and an endoscope receiving port 1014 in which to receive an endoscope 1008, as shown in FIG. 10C. The endoscope may be inserted (along with a lubrication material) into the inner lumen of the overtube. The suction adapter may include one or more seals (not shown) to form a seal around the endoscope once inserted into the endoscope receiving port. The suction source, such as a suction pump, wall suction, etc., forming a suction line 1036 may then be coupled to the suction port (e.g., vacuum port 1016). The control coupled to the vacuum port may include a biased valve against which the control input may be driven to apply suction from the distal end opening of the overtube while the user is activating the control.

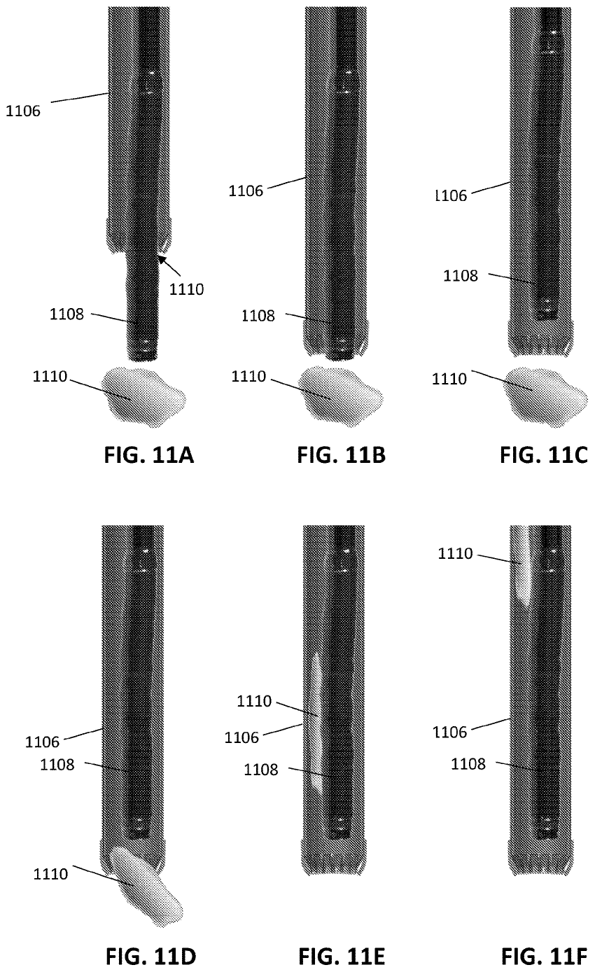

FIGS. 11A-11F illustrates one example of a method of operating an apparatus as described herein. In FIG. 11A the overtube 1106 that is advanced so that an endoscope 1108 that is extended distally out of the distal opening 1110 of the overtube is adjacent to a material to be removed (e.g., clot material 1110). The endoscope may visually confirm location and proximity of the clot material using imaging (e.g., one or more cameras) on the distal end of the endoscope. The overtube 1106 may then be advanced distally towards the clot material 1110, as shown in FIG. 11B, until the distal end region is adjacent to the clot material. The endoscope may then be withdrawn proximally into the lumen of the overtube, as shown in FIG. 11C, and suction may be applied (FIG. 11D), drawing the clot material into the inner lumen of the overtube, as shown in FIGS. 11E and 11F. The endoscope, from within the lumen of the overtube, may confirm that the clot material is drawn in and removed through the overtube inner lumen. The material 1110 may be aspirated up through the lumen of the overtube and alongside the indwelling endoscope 1108; in some cases the material may surround the endoscope as it is aspirated proximally. In FIGS. 11A-11F the overtube distal is tapered, and includes a slightly narrow distal opening 1110 as compared to the inner diameter of the lumen of the overtube.

Although the examples shown above illustrate an apparatus in which an endoscope is relatively centered in the overtube (with the vacuum port as a side exit), alternative configurations may be used. For example, the endoscope may be positioned off-center of the center of the inner lumen of the overtube. For example, the apparatus may be configured so that the endoscope is in the center of the overtube lumen for the mid and distal sections, but it may exit from a (sealed) side exit of the overtube lumen at or near the proximal end. In this configuration, the clot may have a direct suction path along the center of the lumen of the overtube.

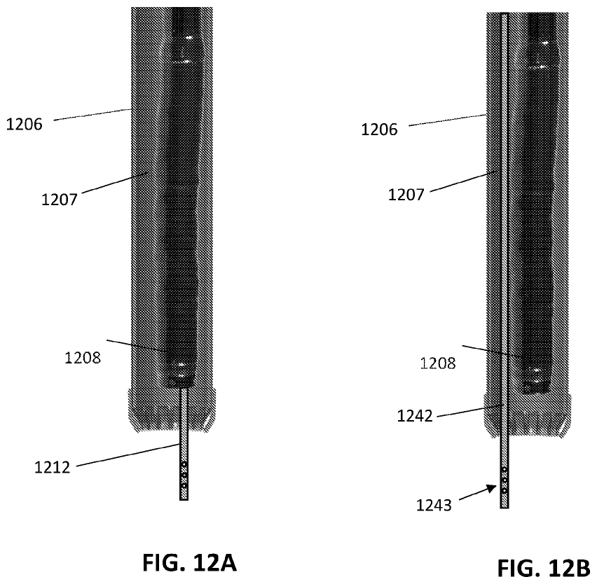

In some examples it may be helpful to apply irrigation with the large bore suction through the overtube, as illustrated in FIG. 12A. In this example, similar to that shown in FIGS. 11A-11F, the apparatus includes an overtube 1206 with an internal suction lumen 1207, and an endoscope 1208. The apparatus (either as part of the endoscope, or as a separate component) may include an irrigation line 1212 (also referred to herein as an irrigator tube) that may extend distally out of the overtube and/or endoscope and may apply irrigation fluid from one or more irrigation outlets. Irrigation may help dislodge material that may be removed by the application of suction from the overtube (in addition, suction may remove the irrigation material). Irrigation may also occur directly out of the internal suction lumen/working channel. Any of these apparatuses may also be configured to apply irrigation from a proximal end of the overtube to flush out clogs and potentially clear the scope.

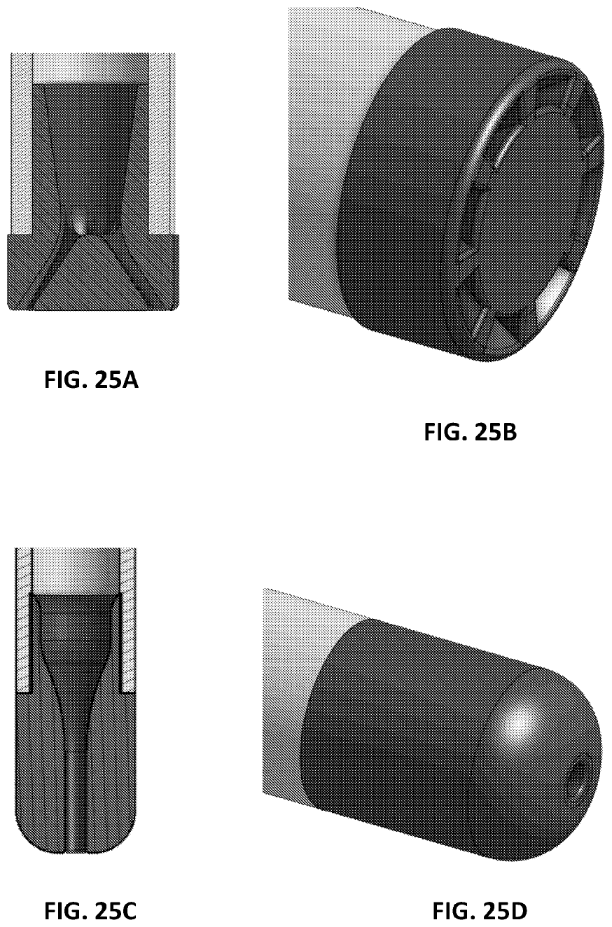

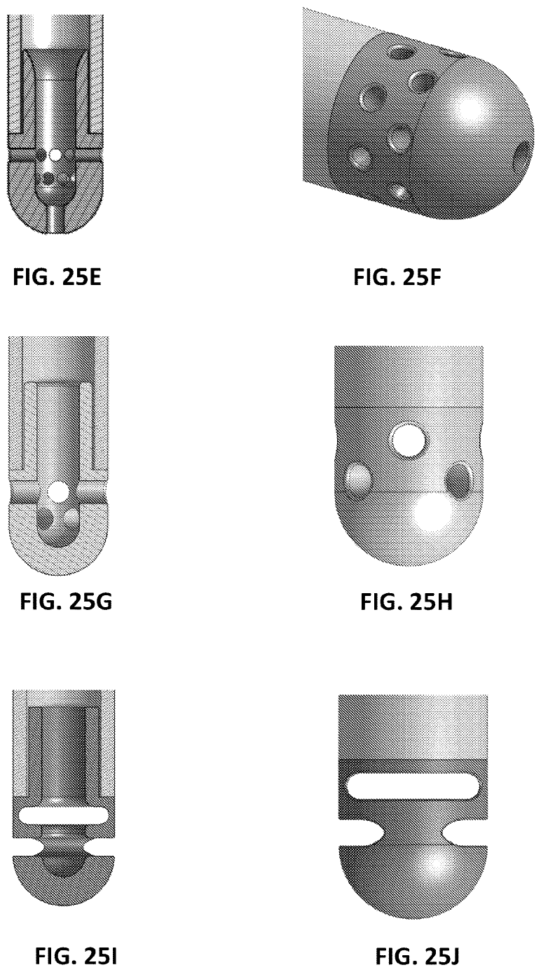

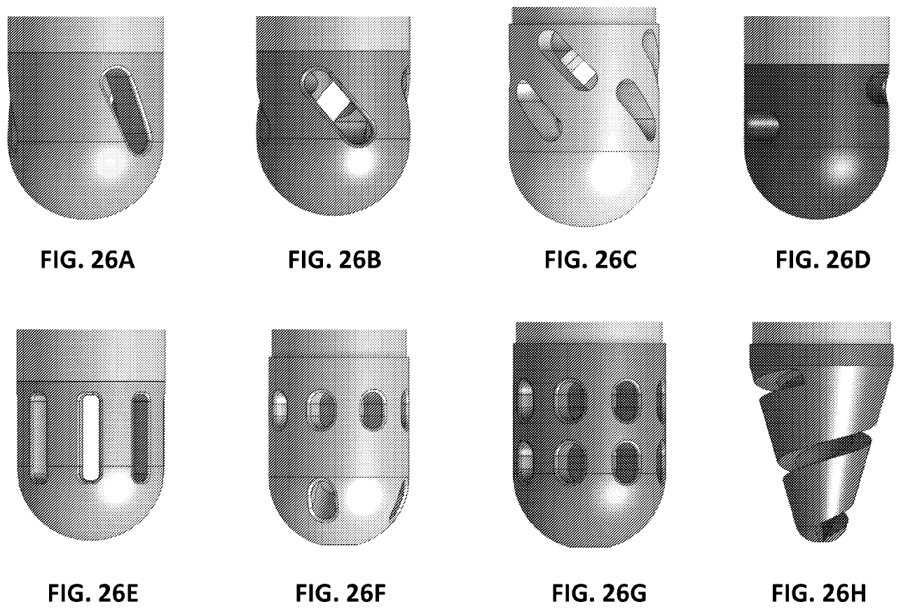

In some examples the irrigator tube 1212 may be separately within the lumen of the overtube, without needing to pass through the endoscope, alternatively a separate irrigator tube 1242 may be used. In general (and as shown in FIGS. 25A-25J and 26A-26H) the irrigator tube may include one or more nozzles 1243 or openings into the irrigator. For example, as shown in FIG. 12B an irrigator tube 1242 may be inserted into the inner lumen 1207 of the overtube 1206 alongside the endoscope 1208 and positioned axially independently of the endoscope. irrigation fluid may be sprayed from the irrigation tube for cleaning the endoscope, disrupting material and/or flushing the endoscope and/or overtube.

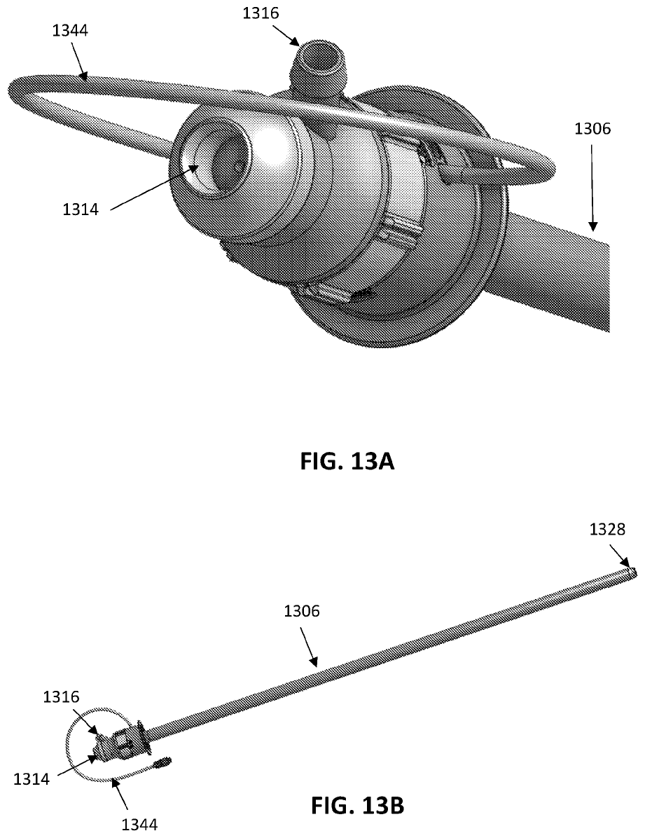

As mentioned, any of these overtubes may be configured as rigidizing overtubes. Alternatively, any of these apparatuses may not be rigidizing overtubes. For example, FIGS. 13A-13B illustrate another example of an apparatus including an overtube having one or more reinforcing layers, but that is not configured to rigidize. FIG. 13A illustrates the proximal end region of the apparatus, showing the suction adapter region coupled to the proximal end of the overtube 1306. The suction adapter region includes a suction port 1316 for coupling to a source of suction, and an endoscope receiving port 1314 for receiving and sealing an endoscope within the suction lumen of the overtube. The apparatus may also include a flush line 1344. In the example shown in FIGS. 13A-13B, the apparatus includes an integrated suction port (shown as a Y-port extending from a side of the apparatus) in the handle region of the overtube apparatus. Any of these features may alternatively or additionally be configured as a rigidizing overtube (including with a built in Y port similar to that shown in FIG. 13B.). The distal end region 1328 may be tapered.

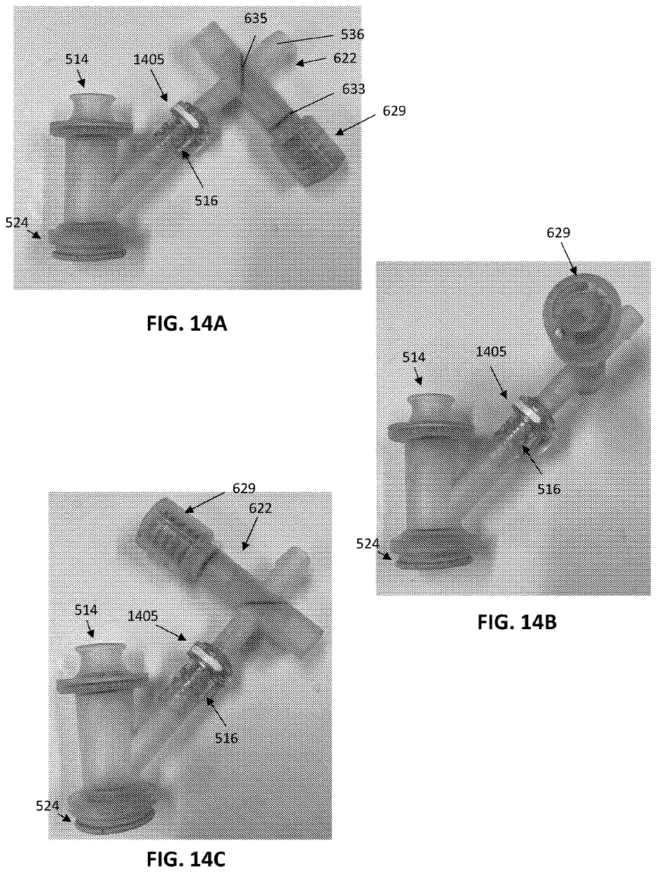

In some examples the control for controlling suction within the overtube may be coupled to the suction adapter portion of the apparatus, as illustrated in FIG. 5A, above. For example, the control may couple to the vacuum port 516 of the apparatus, and in-line with the source of suction. In some cases this connection between the suction line and the apparatus may be a flexible or movable connection, as shown in FIGS. 14A-14B, which illustrates swivel connection 1405 between the adapter's suction port 516. The adapter region may include the endoscope receiving port 514 and coupling region 524. Similarly the controller 622, including (in this example) a trumpet valve as shown in FIGS. 6A-6C may also include a control input 629 (e.g., button, knob, etc.) to open the suction line to apply suction into the lumen of the overtube.

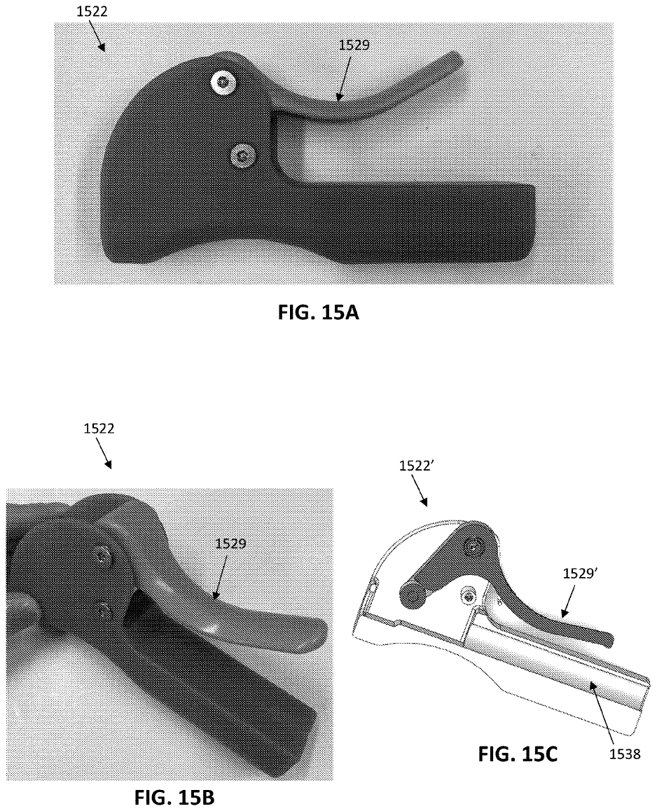

In any of these examples the control may instead by applied to the suction (negative pressure) line connecting the suction port 516 of the overtube (or in some examples, of the suction port of a suction adapter configured to couple to a suction port). The controller may include a valve that is either in-line or parallel to the suction line connecting the source of negative pressure (suction) to the suction port of the apparatus. For example the controller may include a suction line coupling region 1538 configured to couple to the suction line and to close or open (when actuated) to apply suction through the inner lumen of the overtube. In the examples of the controls 1522, 1522′ shown in FIGS. 15A-15C, the control include a lever valve that is configured to cut off the application of suction through as suction line to which it is coupled. In FIG. 15A the actuator (control input 1529) is a lever arm that may be held down to allow suction through a suction line to which the control is attached.

FIG. 15C schematically illustrates another example of a controller 1522′ having a lever or handle 1529′ that can be held down (or squeezed) by the user to allow suction through the overtube lumen when actuated. The control may be biased to return to the closed state when not actuated. In some examples the control may include a lever valve.

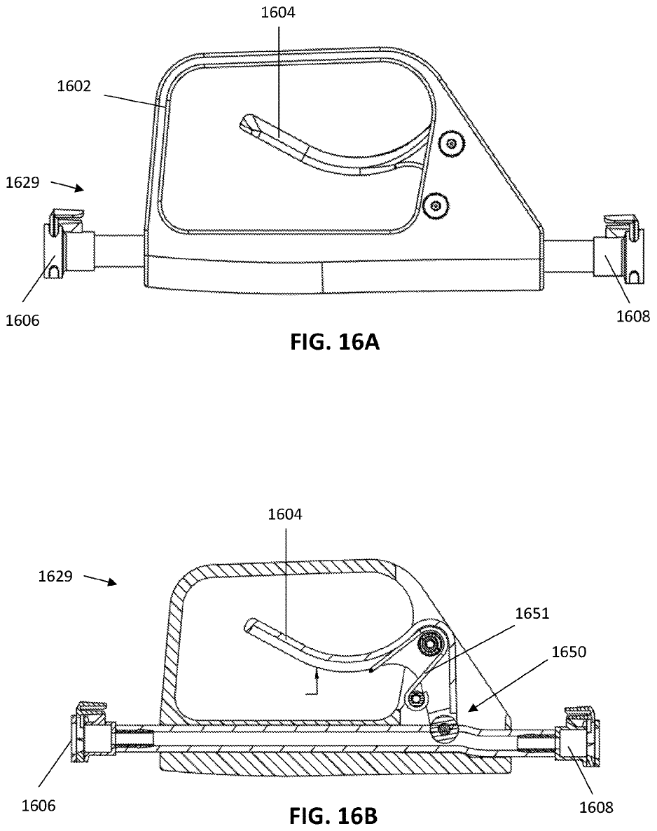

FIGS. 16A-16B illustrate another example of a control 1629 including an actuator (control input 1604) that includes a handle portion 1602 with in which the actuator is also shown as a lever that may be compressed by the user to allow aspiration through the inner lumen of an overtube. For example, as illustrated in FIG. 16B, showing a cross-section through the control of FIG. 16A, the control may be connected in line with the suction line through a suction inlet 1606 and a suction outlet 1608. For example, the apparatus may include a pinch valve 1650 that may be biased (e.g., by a bias, such as a spring 1651) to remain closed until actuated by the user.

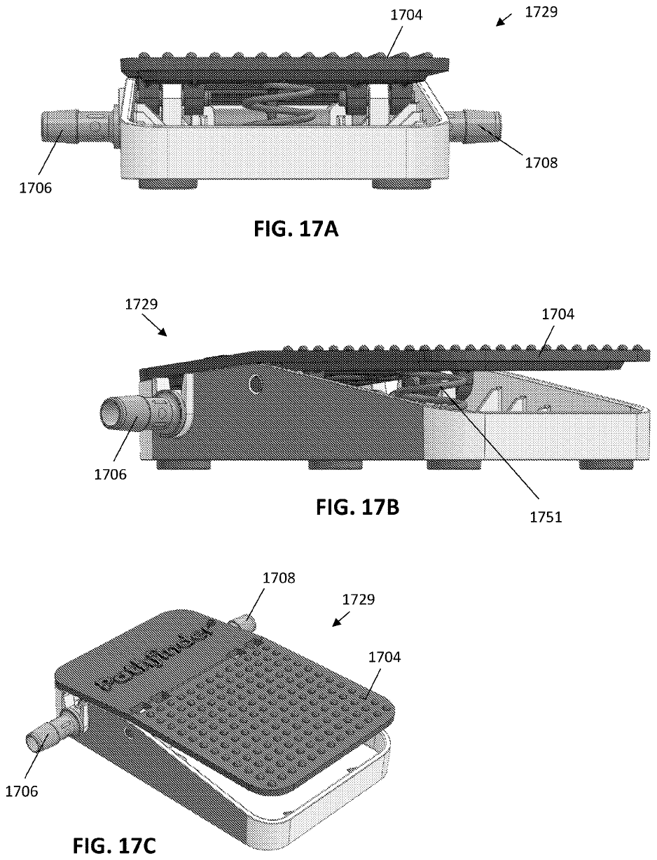

FIGS. 17A-17C illustrate another example of a controller, 1729 including an actuator (control input 1604) that is configured to receive a foot to be compressed 1704, by the user to allow aspiration through the inner lumen of an overtube. For example, as illustrated in FIGS. 17A-17C, the control may be connected in line with the suction line through a suction inlet 1706 and a suction outlet 1708. The controller may include a pinch valve that may be biased 1751 (e.g., by a bias, such as a spring) to remain closed until actuated by the user.

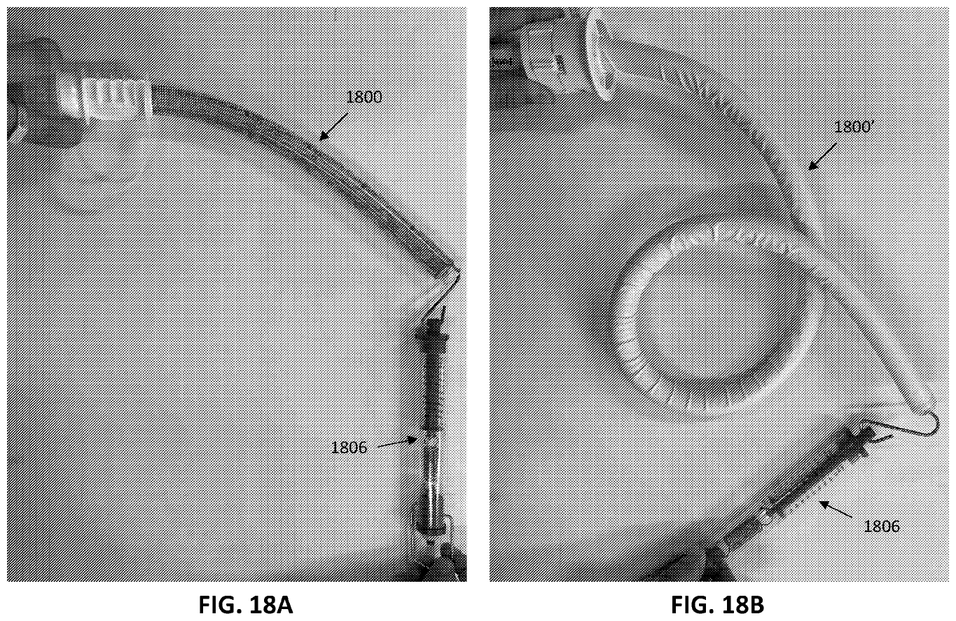

As mentioned above, any of these apparatuses may be configured as a very flexible overtube that is configured to withstand and remain flexible even with a relatively large negative pressure (suction) applied through the lumen of the overtube. FIGS. 18A and 18B illustrate examples of different examples of overtubes to which strain gauges are coupled in order to demonstrate the force applied to bend the flexible elongate member of overtube without breaking.

In FIG. 18A the overtube 1800 includes a reinforcing wire, but is relatively stiff, as compared to the example overtube 1800′ shown in FIG. 18B. In this case, the overtube is both extremely flexible (e.g., has a greater bending flexibility as compared to the overtube shown in FIG. 18A) and has a greater hoop strength under compression sufficient to withstand at least one atmosphere (760 mmHg) or more. A force gauge 1806 is shown attached to the distal end of each overtube to measure the relative force for bending over the length (e.g., to determine bending flexibility). When equivalent bending forces are applied the example overtube 1800′ shown in FIG. 18B is able to bend 360 degrees or more with relative ease.

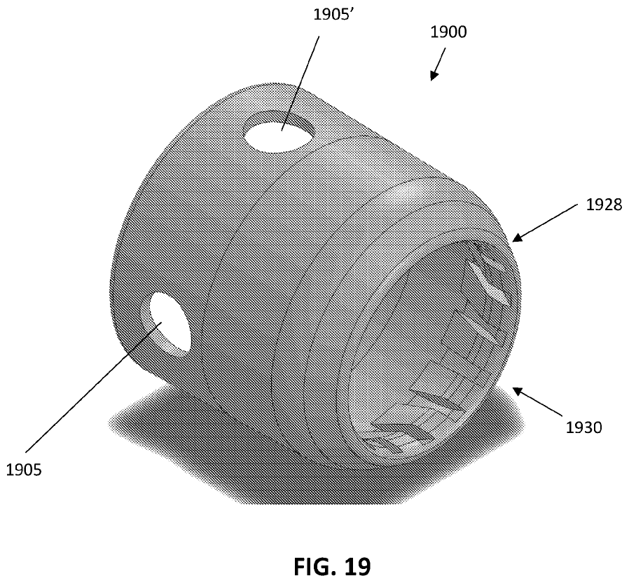

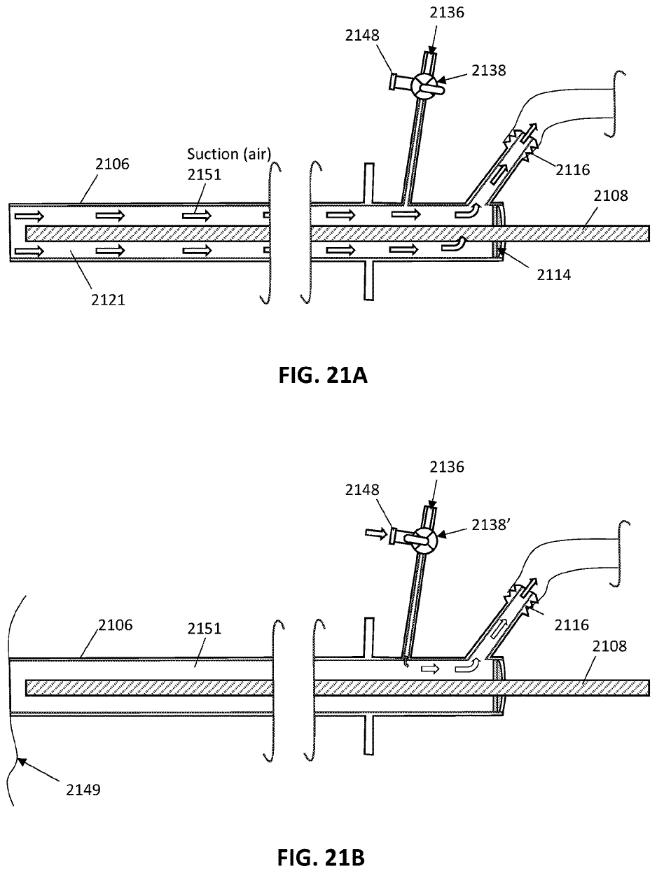

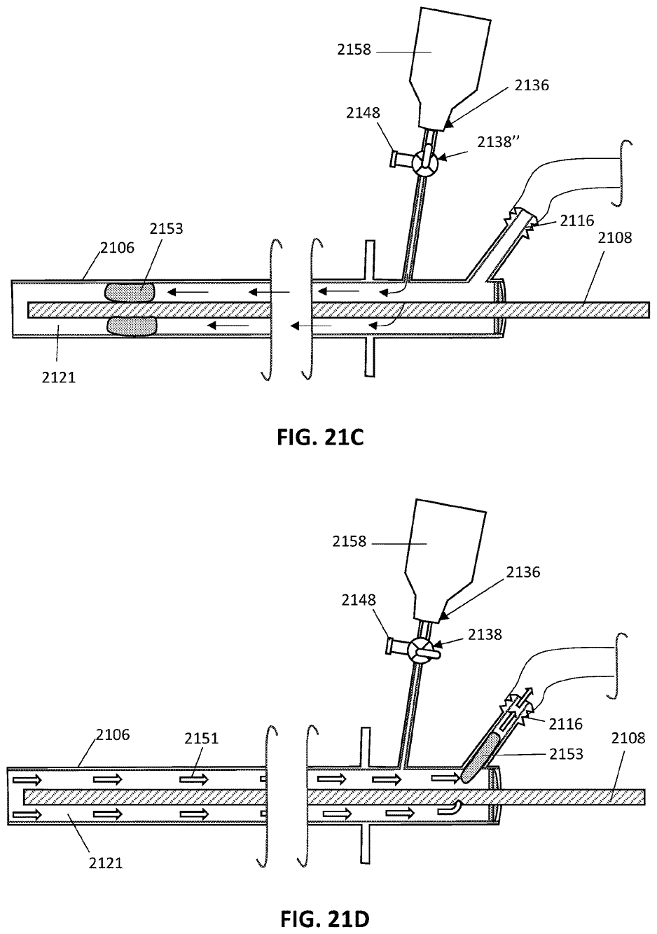

As mentioned above, any of the apparatuses described herein may include one or more openings (e.g., suction release or vacuum release) to prevent vacuum lock of the distal end region (including the opening into the inner lumen of the overtube) when the distal end of the overtube is sucked onto the wall of a body region. FIG. 19 illustrates an example of a distal end region 1900 including a plurality of suction release openings 1905, 1905′ that are arranged laterally and proximal to the distal end opening 1930 into the lumen of the overtube. The distal end opening 1930 is tapered 1928 to have a tapering profile from the outside and also has a narrower opening diameter then the inner diameter of the region of the lumen more proximal to the distal end opening. Alternatively or additionally, these apparatuses may include a relief valve, as illustrated in FIG. 21B.

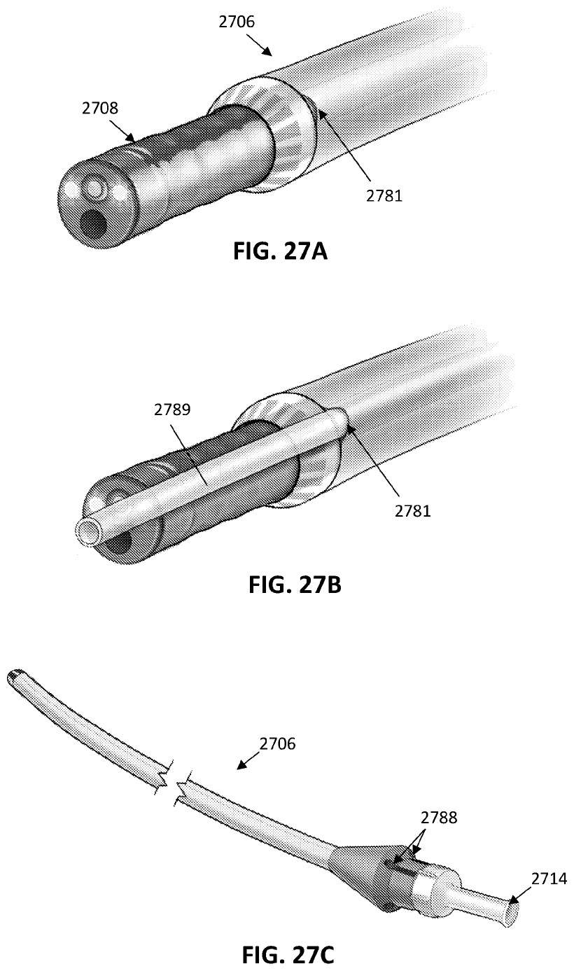

Any of the overtube apparatuses described herein may include one or more external working channels running down the length of the overtube. Examples of external working channels may be found, for example, in U.S. patent application Ser. No. 17/940,906, titled “EXTERNAL WORKING CHANNELS,” and filed on Sep. 8, 2022, and U.S. patent application Ser. No. 18/000,062, titled “RIGIDIZING DEVICES,” and filed on May 26, 2021, each of which is herein incorporated by reference in its entirety. This is illustrated in FIGS. 27A-27C. FIG. 27A shows a distal end region of an overtube 2706 from which an endoscope 2708 is shown extending. The distal end is tapered in this example, as described above. The overtube also includes at least one external working channel 2781, that may be configured to lay flat until an accessory device (e.g., suction tube 2789) is passed through it, as illustrated in FIG. 27B. FIG. 27C illustrates an example of an overtube as described herein including a plurality of (e.g., four) eternal working channels that may be accessed via proximal ports 2788. The proximal end may include an endoscope receiving port 2714 The one or more external working channels may be expandable from the outside surface of the overtube and may include a proximal insertion guide region for inserting one or more device through the external working channel.

### Robotic Apparatuses

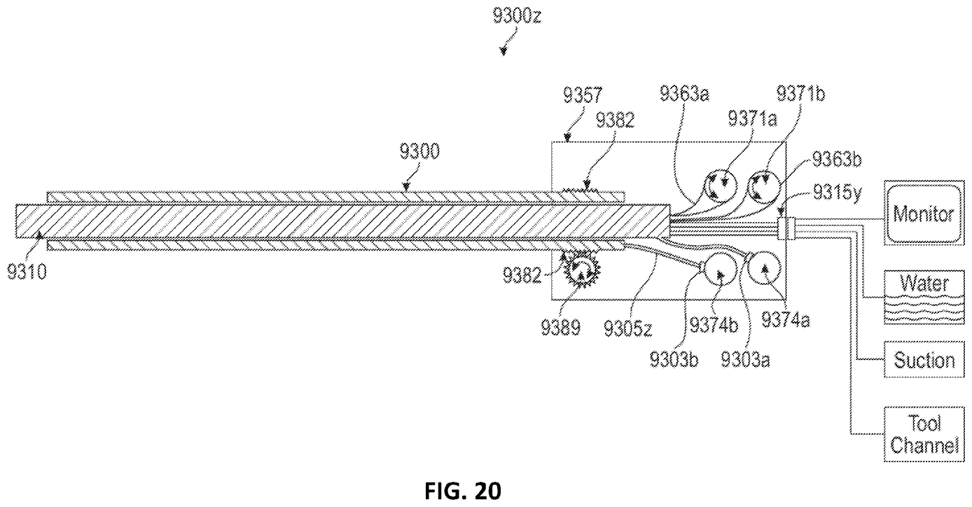

Any of these apparatuses may be configured as robotic apparatuses or for use with robotic apparatuses. In some examples the overtube and endoscope assembly (including the inner endoscope 9310) can be robotically controlled. Any appropriate control subsystem may be used for controlling movement, including steering of the distal ends, advancing and/or retracting the overtube and inner endoscope, and/or rotating the inner endoscope and/or outer overtube. In FIG. 20 an exemplary apparatus 9300z, including an overtube 9300 and inner endoscope 9310 can be robotically controlled or manipulated (e.g., steering, movement, rotation, etc. including in some examples, rigidizing). As shown in FIG. 20, the outer overtube 9300 and the inner endoscope 9310 may be terminated together into a common structure, such as a cassette 9357. The outer overtube 9300 can be movable with respect to the endoscope 9310 by rotation of a disk 9389 that is mounted to the cassette 9357. For example, the disk 9389 can be a pinion, and the outer rigidizing device 9300 may have a rack 9382 including a plurality of small teeth on the outside thereof. Rotating the disk 9389 against teeth 9382 may cause the overtube 9300 to advance forward or backward relative to endoscope 9310. In some examples, the possible movement or translation of the endoscope and/or overtube is limited by the size or design of the cassette 9357.

The cassette 9357 can further include additional disks 9371a, 9371b that may connect to cables 9363a, 9363b respectively, to steer (e.g., bend or deflect) the tip of the endoscope 9310 (and/or outer overtube 9300). Other steering mechanisms (e.g., pneumatics, hydraulics, shape memory alloys, EAP (electro-active polymers), or motors) are also possible. Again, in examples with different steering mechanisms, one or more disks in the cassette 9357 (e.g., disks 9371a, 9371b) may be used to actuate the steering.

The cassette 9357 can further include bellows 9303a, 9303b that may connect to the pressure gap of the endoscope 9310 and the overtube 9300, respectively. Compressing bellows 9303a, 9303b may drive fluid through pressure lines 9305z, causing the pressure in the pressure gap of the endoscope 9310, 9300 to rise, causing the endoscope 9310 and/or the overtube 9300 (in variations for either the endoscope and/or the overtube that are configured to rigidize) to become rigid. Activation of the bellows 9303a, 9303b may be applied sequentially and/or simultaneously. As shown, the cassette 9357 can include eccentric cams 9374a, 9374b to control bellows 9303a, 9303b. Alternatively, one or more linear actuators (e.g., on cassette 9357 or on a drive unit) can be configured to actuate the bellows 9303a, 9303b. As another alternative, the devices 9300, 9310 can be rigidized and de-rigidized through one or more sumps (as described herein) or pressure sources (e.g., via pressure line 9305z).

The cassette 9357 can include a connector 9315y for connecting to additional lumens and/or wiring in the endoscope 9310 and/or overtube 9300. The connector 9315y may include a connection for the delivery of both suction and water to the tip of the endoscope 9310. The connector 9315y may include an electrical connector to connect to a camera mounted to the tip of endoscope 9310 to an external monitor and/or video processing unit.

The connector 9315y may include a mechanical connector that connects to a hollow tube (e.g., working channel) leading all the way to the tip of the inner rigidizing device 9310. By including the connector 9315y, the control of all components of the system 9300z can be performed with the cassette 9357.

In use, these apparatuses may remove material from the body of a patient very efficiently and safely. For example, FIG. 21A illustrates the operation of one example of an apparatus as described herein. In this example, the apparatus includes an overtube 2106 having an elongate body. The overtube may be rigidizing as described above. The proximal end of the overtube includes a suction port 2116 and an endoscope receiving port 2114 with a seal. The apparatus shown in FIGS. 21A-21D shows an endoscope 2108 inserted into the inner lumen of the overtube, forming an annular lumen 2121 as shown. The apparatus also includes port with a three-way valve (e.g., stopcock 2138) having an off position, a first position in which the inner lumen of the overtube is in fluid communication with a rinse or irrigation line 2136 and a second position in which the inner lumen of the overtube is in fluid communication with an air relief port or opening 2148. Thus, in this example, the apparatus includes a manual three-way stopcock 2138 which allows a user to switch between air relief or irrigation. In some examples, irrigation may allow use of a syringe to flush our annular space with water to remove clogs (see FIGS. 21C-21D). In some cases the suction relief 2148 may include a seal (e.g., a tyvek seal) to vent the air to atmosphere, but may block fluids that would come with it.

In FIG. 21A, the device is shown with suction 2151 being applied through the inner lumen of the overtube, from the suction port 2116, which is connected (not shown) to a source or suction and collection. As described above, in use, the distal end of the rigidizing overtube 2016 may be positioned adjacent to a material to be removed with the endoscope 2108 extending distally from distal end. The rigidizing overtube 2106 concentrically surrounds the endoscope 2106 with the overtube and endoscope forming an annular lumen 2121. The apparatus may be maneuvered within the body by controlling the rigidity of the overtube to assist in navigation; the endoscope may be steered and driven distally while the overtube is rigid, then the overtube may be made flexible and driven over the endoscope, then rigidized again to retain the shape, allowing additional navigation distal to the overtube. Once the endoscope is in position, e.g., visualizing material to be removed or a region to be rinsed and/or treated, the endoscope may be retracted slightly proximally into the overtube, so that the distal end of the endoscope is within a distal end region of the rigidizing overtube, as shown in FIG. 21A. This may be done with the overtube rigidized. Suction may then be applied through the annular lumen to aspirate the material into the annular lumen and around the endoscope.

In some cases the overtube may become locked onto a wall 2149 of the lumen, as shown in FIG. 21B. in this case, as mentioned above, openings on the distal end region may help release the lock from the wall. Alternatively in some cases the relief valve (e.g., air relief port 2148) may be used. In FIG. 21B, the valve 2138′ is shown with the stopcock in the second position so that air is vented from outside of the body, which may rapidly release the vacuum lock on the wall.

FIGS. 21C-21D illustrate an example in which the annular lumen 2121 becomes clogged (e.g., with material 2153) and this clog may be removed by the application of wash fluid 2158 (e.g., from a syringe) applied through the wash port 2136 that is controlled by the three-way valve 2138″ (shown in the first, wash, position in FIG. 21C). For example, in FIG. 21C the clog 2158 within the annular lumen of the device 2121 is loosened or removed by the application of rinse fluid (e.g., saline, etc.) 2158 when the three-way valve opens the wash port (e.g., rinse port) 2136. The rinse fluid may be applied by a syringe in some examples. The clog may be driven distally out of the device and may be removed by once more applying suction 2151 through the suction port 2116, as shown in FIG. 21D. In FIG. 21D the 3-way stopcock 2138 is once more in the closed position.

In any of these methods, a clog may also or alternatively be removed by moving the endoscope within the inner lumen. For example, the endoscope may be moved axially (proximally/distally). In some cases the endoscope may be moved axially between about 1 mm and 3 cm or more, e.g., between about 1 mm and about 3 cm, between about 1 mm and 2.5 cm, between about 1 mm and 2 cm, between about 1 mm and 1.5 cm, between about 1 mm and 1 cm, between about 1 mm and 7 mm, between about 1 mm and 5 mm, etc.). The endoscope may be moved back and forth in a linear oscillation, e.g., at between about 1 Hz and 50 Hz (e.g., between about 1 Hz and 40 Hz, between about 1 Hz and 30 Hz, between about 1 Hz and 20 Hz, between about 1 Hz and 10 Hz, etc.). Moving the endoscope within the lumen of the overtube while applying suction may help prevent or remove a clog. Alternatively or additionally the endoscope may be rotated within he lumen.

In any of these apparatuses a clot may be detected when fluid, which may be applied before and/or during the application of suction, is not removed. This may be detected or seen by looking at a collector in line with the source of suction.

As mentioned above, fluid may be applied during operation of these apparatuses to loosen and/or remove material from the body region, including impacted fecal matter, mucus, etc. Fluid may be applied via one or more fluid channels that may be part of the overtube, part of the endoscope, or part of a separate tool inserted into either the overtube and/or endoscope. Fluid may be applied by a fluid pump at any appropriate flow rate. In some cases fluid may be applied in a pulsatile manner, e.g., with a frequency of between about 0.1 to 200 Hz (e.g., between about 0.5-150 Hz, etc.). In any of these methods and apparatuses gas (e.g., air) may be included with the applied fluid to disrupt the material to be removed.

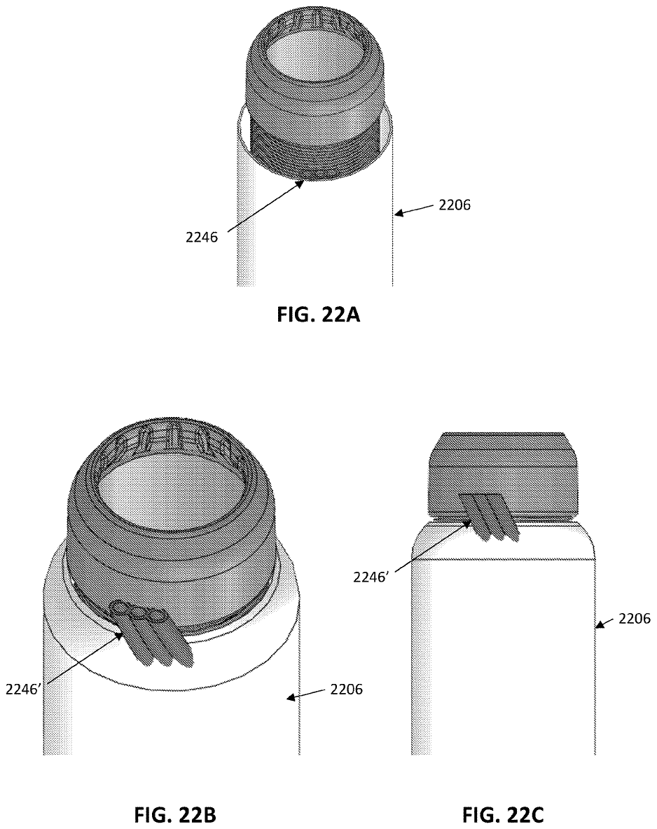

FIGS. 22A-22C illustrate examples in which fluid is applied through a fluid line of the overtube. In FIG. 22A the overtube 2206 includes three fluid lines 2246 that include outlets that are slightly proximal to the distal end of the overtube. In this example, the fluid outlets are positioned radially on one side of the overtube. In some cases, as shown in FIG. 22B, the outlets of the fluid lines (three are shown) 2246′ may extend slightly distally from the overtube (though still proximal to the distal end of the overtube) and may be angled or arranged at an angle to the long axis of the overtube, as shown in FIGS. 22B-22C.

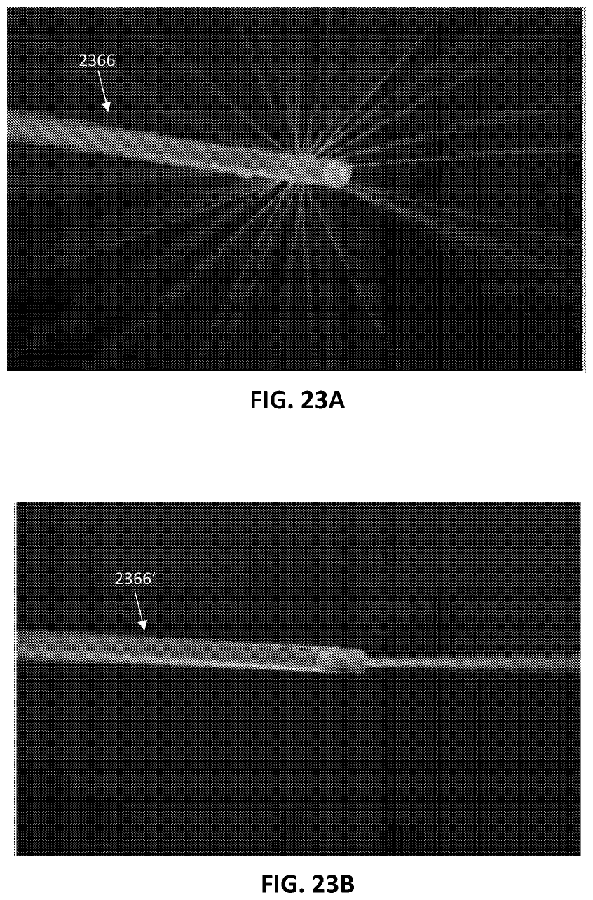

Alternatively or additionally, fluid (e.g., wash fluid or irrigation fluid) may be applied from the distal end region of an irrigator tube that may be inserted through the endoscope and/or through the overtube. The direction of fluid from any of these fluid lines, including irrigators and integrated fluid lines, may be controlled by a nozzle region at the distal end. In some cases it may be particularly beneficial to provide a radial spray, as illustrated in FIG. 23A. FIG. 23A shows an irrigator tube 2366 configured as a radial spray irrigator tube in which the distal end region is configured as a nozzle having multiple annular openings to form a radial spray, a shown. In contrast, FIG. 23B shows an example of an irrigator tube 2366′ in which a linear spray is emitted, out of a single nozzle opening at the distal end. This may produce a single jet irrigation spray, as shown.

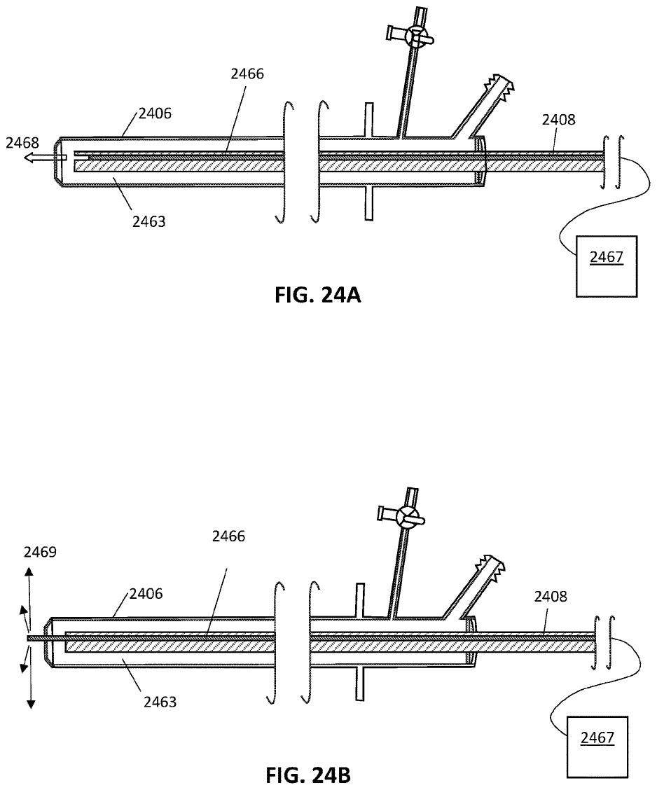

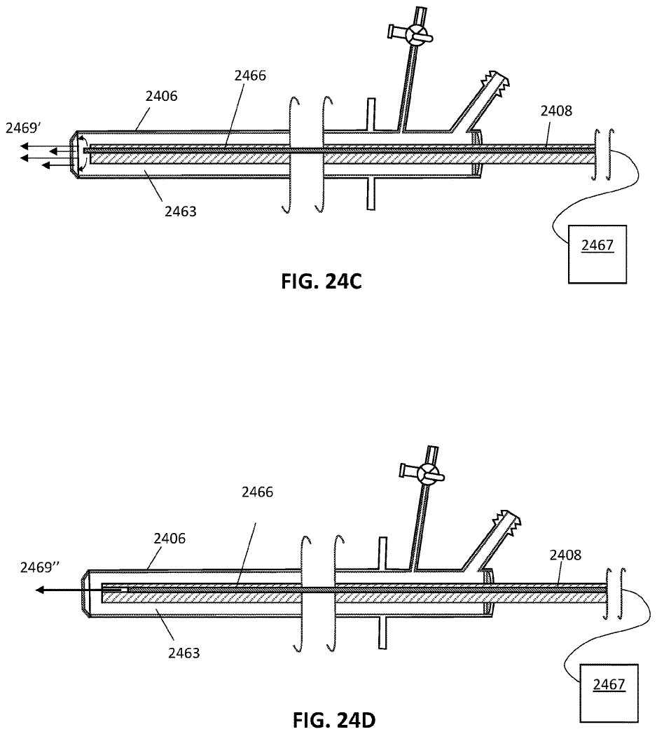

In some cases, the apparatus may be configured to be operated so that a single irrigator tube may be used as both a radial spray and a longitudinal (e.g., distal) jet, by adjusting the relative position of the irrigator tube out of the distal end of the endoscope and/or overtube, as illustrated in FIGS. 24A-24D. For example, in FIG. 24A the radial spray irrigator 2466 is being advanced 2468 distally through the endoscope 2408 which has been retracted slightly proximally within the overtube 2406 lumen, forming an annular lumen 2463, as described above. The irrigator tube 2466 is coupled to a source of irrigation fluid 2467, which may be a pump or syringe. Once the distal end of the irrigator tube 2466 is extended distally from out of both the endoscope and the overtube 2406, irrigation fluid may be sprayed out radially 2469 from the distal end (e.g., nozzle), as shown in FIG. 24B.

The radial spray irrigator tube may also be used to apply a longitudinal stream, by withdrawing the radial spray irrigator tube 2466 proximally into either the overtube 2406, as shown in FIG. 24C, resulting in a large, distally directed stream 2469′, as shown. Alternatively, in some examples, the irrigator tube 2466 may be retracted so that the radial spray nozzle region at the distal end is retracted into the endoscope 2408, as shown in FIG. 24D, resulting in a distal stream 2469″.

FIGS. 25A-25J illustrate examples of different variations of irrigator tube distal ends, configured as nozzle regions. FIGS. 25A-25B show cross-sectional and side perspective views, respectively, of a first distally-directed nozzle. FIGS. 25C-25D show cross-sectional and side perspective views, respectively, of a distally (e.g., longitudinally) directed spraying nozzle. FIGS. 25E-25F show cross-sectional and side perspective views, respectively, of a combination radial and distally spraying nozzle. FIGS. 25G-25H show cross-sectional and side perspective views, respectively, of a radially spraying nozzle. FIGS. 251-25J show cross-sectional and side perspective views, respectively, of an example of a nozzle configured to emit a radial spay (delivered as a fan-shaped spray, rather than discrete streams. These nozzle regions may be formed on the irrigator tube, or they may be attached to a distal end of the irrigator tube. In some cases the nozzle region may be formed by molding, printing (3D printing), laser cutting, etc.

FIGS. 26A-26H show side views of various additional examples of nozzle regions of irrigator tubes as described herein. Thes different irrigator tubes may be used to achieve a variety of different spraying patterns. Any of these irrigator tubes may converted to a forward (e.g. laterally) streaming irrigator tube by withdrawing it into the overtube and/or endoscope, as described in FIGS. 24C-24D, above.

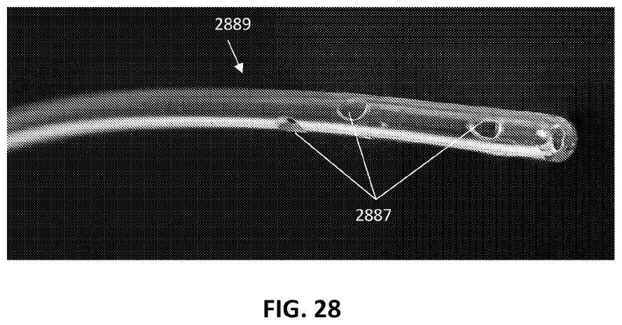

In addition to the irrigator tubes described herein these other accessories may be included as well. For example, any of these apparatuses may be used with (e.g., in conjunction with) one or more suction tubes, such as shown in FIG. 28, and described above. For example, a suction tube 2889 may be inserted through the endoscope (e.g., a working channel of the endoscope) and/or the overtube, including a working channel that is within the inner lumen and/or an external working channel. The suction tube 2889 may include one or more openings 2887 into the apparatus, as shown in FIG. 28. In this example, the suction tube is a 6 mm disposable suction tube.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/31 2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of removing material from the body of a patient, the method comprising:
positioning a distal end of a rigidizing overtube adjacent to the material with an endoscope extending distally from distal end, wherein the rigidizing overtube concentrically surrounds a length of the endoscope so that the overtube and endoscope form an annular lumen;
retracting a distal end of the endoscope proximally into a distal end region of the rigidizing overtube;
and applying suction through the annular lumen to aspirate the material into the annular lumen and around the endoscope.

2. The method of claim 1, further comprising converting the rigidizing endoscope from a more flexible configuration when positioning the distal end adjacent to the material, to a more rigid configuration before retracting the endoscope proximally into the distal end region of the rigidizing overtube.

3. The method of claim 1, further comprising visualizing the material using the endoscope while applying suction.

4. The method of claim 1, further comprising applying irrigation distal to the distal end of the overtube.

5. The method of claim 4, wherein applying irrigation comprises applying a spray of fluid from a fluid line of the overtube.

6. The method of claim 4, wherein applying irrigation comprises extending an irrigator tube distally out of the distal end of the endoscope and applying irrigation into the body from the irrigator tube.

7. The method of claim 5, wherein the irrigator comprises a radial irrigator.

8. The method of claim 4, wherein applying irrigation comprises switching between applying radial irrigation and distal irrigation by extending or retracting a radial irrigation tube relative to the distal end of the endoscope.

9. The method of claim 1, further comprising moving the endoscope axially within the overtube while applying suction.

10. The method of claim 1, further comprising moving the endoscope axially within the overtube while positioning the overtube.

11. The method of claim 1, further comprising steering the distal end region of the endoscope independently of the overtube.

12. The method of claim 1, wherein positioning the overtube comprises rigidizing the overtube while advancing and steering the endoscope distally from the overtube, de-rigidizing the overtube, and advancing the overtube distally over the endoscope.

13. The method of claim 1, wherein a proximal end of the overtube is movably sealed around the endoscope.

14. The method of claim 1, wherein the annular lumen has a cross-sectional area of between 40 mm² and 200mm².

15. The method of claim 1, wherein positioning comprises positioning the overtube within the patient's gastrointestinal tract.

16. The method of claim 1, wherein removing the material from the body comprises removing one or more of: fecal matter, blood and food debris.

17. The method of claim 1, further comprising unclogging the annular lumen by repeatedly moving the endoscope distally and proximally within the overtube while apply suction through the annular lumen.

18. The method of claim 1, further comprising releasing the overtube from a wall of the body by opening an air relief valve in fluid communication with the annual lumen while applying suction through the annular lumen.

19. A method of removing a material from the body of a patient, the method comprising:
positioning a distal end of an endoscope adjacent to the material;
positioning a distal end of an overtube extending over the endoscope adjacent to the distal end of the distal end of endoscope with the overtube in a flexible configuration so that the endoscope transitions from an extended configuration extending distal to the overtube to a retracted configuration, wherein the endoscope distal end is recessed within the distal end region of the overtube, so that the overtube concentrically surrounds the endoscope forming an annular lumen;
converting the overtube from the flexible configuration to a more rigid configuration; and
applying suction through the annular lumen to aspirate the material into the annular lumen and around the endoscope.

20. A method of removing a material from the body of a patient, the method comprising:
positioning a distal end of an endoscope adjacent to the material;
advancing a distal end of an overtube distally over the endoscope so that the distal end of the overtube is adjacent to the material while the overtube is in a flexible configuration, and positioning the distal end of the endoscope within a distal end region of the overtube to allow visualization out of the distal end of the overtube by the endoscope, so that the overtube concentrically surrounds the endoscope forming an annular lumen;
converting the overtube from the flexible configuration to a more rigid configuration; and
applying suction through the annular lumen to aspirate the material into the annular lumen and around the endoscope.

21.-56. (canceled)
